(19) United States
(12) Patent Application Publication
(10) Pub. No.: US 2010/0094339 A1
(43) Pub. Date: Apr. 15, 2010

(54) COATING COMPOSITIONS

(76) Inventor: Joshua Stopek, Yalesville, CT (US)

Correspondence Address: Tyco Healthcare Group LP, 60 MIDDLETOWN AVENUE, NORTH HAVEN, CT, 06473, US

(21) Appl. No.: 12/446,844

(22) PCT Filed: Dec. 20, 2007
(86) PCT No.: PCT/US07/26003

§ 371 Date: Apr. 23, 2009

Related U.S. Application Data
(60) Provisional application No. 60/876,808, filed on Dec. 22, 2006.

(51) Int. Cl. A61L 17/00; C08K 5/101

(52) U.S. Cl. 606/231; 524/318

(57) ABSTRACT

The present disclosure provides compositions suitable for forming medical devices and coatings for medical devices, including sutures, which include a copolymer, at least one phosphorylcholine, and a fatty acid component. Sutures and other medical devices possessing such coatings are also provided.

### CROSS-REFERENCE TO RELATED APPLICATIONS

This application claims the benefit of and priority to U.S. Provisional Patent Application No. 60/876,808, filed Dec. 22, 2006, the entire disclosure of which is incorporated by reference herein.

### TECHNICAL FIELD

Absorbable materials fabricated from compositions including caprolactone containing copolymers, phosphorylcholines, and a fatty acid component including fatty acids, salts of fatty acids, or salts of esters of fatty acids, are provided. In embodiments, the compositions of the present disclosure may be used to form medical devices or as coatings for surgical articles, including coatings for sutures.

### BACKGROUND

Synthetic absorbable multifilament sutures such as DEXON™, VICRYL®, and POLYSORB™, commercially available from Ethicon, Inc. (Somerville, N.J.), and United States Surgical Corporation (Norwalk, Conn.), are well known in the industry.

Suture coatings for synthetic absorbable sutures containing caprolactone are also known. See, for example, U.S. Pat. Nos. 4,624,256; 4,190,720; 4,582,052; 4,605,730; 4,700,704; 4,705,820; 4,788,979; 4,791,929; 4,994,074; 5,047,048; 5,100,433; 5,133,739; and 5,352,515. Suture coatings containing esters of fatty acids and/or salts of fatty acids are also known. See, for example, U.S. Pat. Nos. 5,716,376, 5,032,638, 4,711,241, 4,705,820, 4,201,216 and 4,027,676.

One important feature of a suture coating is its ability to enhance the suture's handling characteristics, such as surgeon's throw, lubricity, knot run down and/or knot security. Although commercially available surgical sutures such as POLYSORB™ have excellent handling characteristics, it would be advantageous to provide a coated suture exhibiting even better surgeon's throw, lubricity, knot run down, and/or knot security properties.

### SUMMARY

In an embodiment of the present invention, there is provided a surgical suture coating including a copolymer comprising a predominant amount of epsilon-caprolactone and a minor amount of at least one other copolymerizable monomer, at least one phosphorylcholine-containing polymer, and a fatty acid component selected from the group consisting of fatty acid salts and salts of fatty acid esters.

In embodiments of the surgical suture coating, the copolymer may comprise from about 70 to about 98 weight percent epsilon-caprolactone derived units. In other embodiments of the surgical suture coating, the copolymer may comprises from about 80 to about 95 weight percent epsilon-caprolactone derived units.

In an embodiment of the surgical suture coating, the at least one other copolymerizable monomer comprises glycolide. In one aspect, the at least one phosphorylcholine-containing polymer comprises a phosphorylcholine selected from the group consisting of 2-methacryloyloxyethyl phosphorylcholine (MPC), 2-acryloyloxyethyl phosphorylcholine, derivatives thereof, and combinations thereof. In an embodiment, the salt of a fatty acid comprises polyvalent metal ion salts of C₆ and higher fatty acids. In one embodiment, the salt of a fatty acid is selected from the group consisting of calcium stearate, magnesium stearate, barium stearate, aluminum stearate, zinc stearate, calcium palmitate, magnesium palmitate, barium palmitate, aluminum palmitate, zinc palmitate, calcium oleate, magnesium oleate, barium oleate, aluminum oleate, and zinc oleate. In one aspect of the invention, the salt of a fatty acid ester comprises a salt of lactylate esters of C₁₀ or greater fatty acids. In an embodiment the salt of lactylate esters of C₁₀ or greater fatty acids is selected from the group consisting of magnesium stearoyl lactylate, aluminum stearoyl lactylate, barium stearoyl lactylate, zinc stearoyl lactylate, calcium palmityl lactylate, magnesium palmityl lactylate, aluminum palmityl lactylate, barium palmityl lactylate, or zinc palmityl lactylate, calcium oleyl lactylate, magnesium oleyl lactylate, aluminum oleyl lactylate, barium oleyl lactylate, and zinc oleyl lactylate.

In one embodiment, the suture coating also includes a solvent selected from the group consisting of methylene chloride, chloroform, N,N-dimethylformamide, N-methylpyrrolidone, methanol, ethanol, n-propanol, isopropanol, hexane, heptane, cyclohexane, tetrahydrofuran, dipropyl ether, dioxolane, methyl ethyl ketone, methyl isobutyl ketone, toluene, xylene, acetonitrile, ethyl acetate, butyl acetate, and combinations thereof.

In one embodiment, the components of the coating are applied separately in multiple layers, while in another embodiment, the components of the coating are applied as a single layer. In an embodiment, the suture coating includes a bioactive agent.

In one embodiment, a suture is provided having a coating including a copolymer comprising a predominant amount of epsilon-caprolactone and a minor amount of at least one other copolymerizable monomer, at least one phosphorylcholine-containing polymer, and a fatty acid component selected from the group consisting of fatty acid salts and salts of fatty acid esters. In an embodiment of the suture, the coating is present in an amount from about 0.3% to about 10% by weight of the suture.

In an embodiment, there is provided a surgical suture coating comprising a copolymer comprising a glycolide, at least one phosphorylcholine-containing polymer, and a fatty acid component selected from the group consisting of fatty acid salts and salts of fatty acid esters.

It is to be understood that both the foregoing general description and the following detailed descriptions are exemplary and explanatory only and are not restrictive of the invention as claimed. Additional features and advantages of the invention will be set forth in part in the description which follows, and in part will be obvious from the description, or may be learned by practice of the invention.

### DESCRIPTION OF THE DRAWINGS

Various embodiments of the present disclosure will be described herein below with reference to the figures wherein:

FIG. 1 is a perspective view of a coated suture of the present disclosure attached to a needle as described herein.

### DETAILED DESCRIPTION

In embodiments, compositions useful in forming the aforementioned articles and coatings thereon include copolymers, phosphorylcholines, and an optional fatty acid component. In some embodiments, the fatty acid component may be present as a predominant component. As used herein, a “predominant component” includes a component which is present in an amount greater than about 50 weight percent. Thus, a “minor component” includes a component which is present in an amount up to about 50 weight percent. In accordance with the present disclosure, where a composition of the present disclosure includes a fatty acid component as a predominant component, the minor component may be a combination of phosphorylcholines and copolymers.

In embodiments, the copolymers may include caprolactone. Suitable caprolactone containing copolymers include copolymers which may be synthesized by polymerization techniques within the purview of those skilled in the art. In embodiments, the caprolactone containing copolymer may be obtained by polymerizing a major amount of epsilon-caprolactone and a minor amount of at least one other copolymerizable monomer or mixture of such monomers in the presence of a polyhydric alcohol initiator. In other embodiments, caprolactone containing copolymers which may be utilized include “star” copolymers obtained by polymerizing a major amount of epsilon-caprolactone and a minor amount of another bioabsorbable monomer polymerizable therewith, optionally in the presence of a polyhydric alcohol initiator.

Suitable monomers which can be copolymerized with epsilon-caprolactone include alkylene carbonates such as trimethylene carbonate, tetramethylene carbonate, dimethyl trimethylene carbonate; dioxanones; dioxepanones; absorbable cyclic amides; absorbable cyclic ether-esters derived from crown ethers; hydroxyacids capable of esterification, including both alpha hydroxy acids (such as glycolic acid and lactic acid) and beta hydroxyacids (such as beta hydroxybutyric acid and gamma hydroxyvaleric acid); polyalkyl ethers (such as polyethylene glycol and polypropylene glycol and combinations thereof); with glycolide being useful in some embodiments.

Suitable polyhydric alcohol initiators which may be utilized in forming the caprolactone containing copolymers include glycerol, trimethylolpropane, 1,2,4-butanetriol, 1,2,6-hexanetriol, triethanolamine, triisopropanolamine, erythritol, threitol, pentaerythritol, ribitol, arabinitol, xylitol, N,N,N′,N′-tetrakis(2-hydroxyethyl)ethylenediamine, N,N,N′,N′-tetrakis(2-hydroxypropyl)ethylenediamine, dipentaerythritol, allitol, dulcitol, glucitol, altritol, iditol, sorbitol, mannitol, inositol, and the like, with mannitol being useful in some embodiments.

The polyhydric alcohol initiator may be generally employed in small amounts, e.g., from about 0.01 to about 5 weight percent of the total monomer mixture, in embodiments from about 0.1 to about 3 weight percent of the total monomer mixture.

The polymerization of the above monomers with caprolactone contemplates all of the various types of monomer addition, for example, simultaneous, sequential, simultaneous followed by sequential, sequential followed by simultaneous, and the like.

The caprolactone containing copolymers can contain from about 70 to about 98 weight percent epsilon-caprolactone derived units, in embodiments from about 80 to about 95 weight percent epsilon-caprolactone derived units, the balance of the copolymer being derived from the other copolymerizable monomer(s).

In embodiments, the copolymerizable monomer may be glycolide, optionally in combination with other monomer(s).

In other embodiments, any of the monomers identified above as suitable as copolymerizable with epsilon caprolactone may be utilized without caprolactone; i.e., they may be used as homopolymers or combined with monomers other than caprolactone to form a copolymer for use in accordance with the present disclosure.

In yet other embodiments, the copolymers utilized in accordance with the present disclosure may be made at least in part from a polyoxyalkylene block copolymer. Suitable polyoxyalkylene block copolymers include those having an A-B or A-B-A structure wherein “A” is a block made from repeating units of the formula —O(CH₂)ₙ— where n is from 1 to 4 and “B” is a block made from repeating units that are different from the repeating units in the A block and are selected from groups of the formula —O(CH₂)ₙ— where n is from 1 to 4. In particularly useful embodiments, a co-polymer designated as “PEO-PPO-PEO”, wherein “PEO” denotes a block of repeating units of the formula —OCH₂CH₂— and “PPO” denotes a block of repeating units of the formula —OCH₂CH₂CH₂—. Particularly useful are triblock copolymers of the formula HO(C₂H₄O)ₐ(C₃H₆O)_{b}(C₂H₄O)_{c}H wherein a and c are independently from 1-150 units and b ranges from 10-200 units, with the overall molecular weight ranging from 1,000 to 50,000 daltons. Such polyoxyalkylene block copolymers may be, in embodiments, referred to by those skilled in the art as “poloxamers”. Suitable poloxamers include those where a equals c and b ranges from 10-200 units.

Examples of polyoxyalkylene block copolymers which may be utilized to form the copolymer utilized in forming the compositions of the present disclosure include poloxamers sold under the trade names PLURONIC® (BASF Corp.) or SYNPERONIC® (ICI). PLURONIC® copolymers are identified by a specific letter-number combination. The alphabetical designation describes the physical form of the product: ‘L’ for liquids, ‘P’ for pastes, ‘F’ for solid forms. The first digit (two digits in a three-digit number) in the numerical designation, multiplied by 300, indicates the approximate molecular weight of the hydrophobic component (propylene oxide). The last digit, when multiplied by 10, indicates the approximate hydrophilic (ethylene oxide) content of the molecule as a percentage by weight. Thus, for example, PLURONIC® F68 is a solid material. The molecular weight of the hydrophobic (propylene oxide) component is approximately 1800 (6×300). The hydrophilic (ethylene oxide) component represents approximately 80% of the molecule by weight (8×10).

Poloxamers can be roughly divided into 3 main categories, each of which may be useful in making the copolymer component of the compositions of the present disclosure, namely emulsion forming, micelle forming, and water soluble poloxamers. Various factors which determine poloxamer characteristics and behavior include the molecular weight, PPO:PEO ratio, temperature conditions, concentration, and presence of ionic materials. There is thus a wide range of characteristics in existing commercially available poloxamers which can be exploited in formulating the compositions of the present disclosure, especially where the composition further includes a medicinal agent or other bioactive agent.

In embodiments, a suitable poloxamer which may be utilized in the copolymer component of a composition of the present disclosure includes a polyoxyethylene-polyoxypropylene triblock copolymer known as poloxamer 188, sold under the trade name PLURONIC® F68 by BASF (Parsippany, N.J.). Other poloxamers which may be utilized in the compositions of the present disclosure include poloxamer 403 (sold as PLURONIC® P123), poloxamer 407 (sold as PLURONIC® P127), poloxamer 402 (sold as PLURONIC® P122), poloxamer 181 (sold as PLURONIC® L61), poloxamer 401 (sold as PLURONIC® L121), poloxamer 185 (sold as PLURONIC® P65), and poloxamer 338 (sold as PLURONIC® F108).

The polyoxyalkylene block copolymers may, in some embodiments, be reacted with additional biocompatible, biodegradable monomers to form the copolymer. Suitable monomers which may be reacted with the polyoxyalkylene block copolymers include, for example, alpha-hydroxy acids, lactones, carbonates, esteramides, anhydrides, amino acids, orthoesters, alkylene alkylates, alkylene oxides, biodegradable urethanes, and combinations thereof. Specific examples of suitable biocompatible, biodegradable monomers which may be added to the poloxamer include glycolide, lactide, hydroxybutyric acid, hydroxyvaleric acid, caprolactone, trimethylene carbonate, dimethyl trimethylene carbonate, p-dioxanone, and combinations thereof. These monomers, alone or in combination, can constitute up to about 90% to by total weight of the copolymer component, in embodiments from about 10% to about 75% by total weight of the copolymer component, in other in embodiments from about 30% to about 65% by total weight of the copolymer component, with the polyoxyalkylene block copolymer making up the balance of the copolymer component. It should, of course, be understood that the other monomers may be reacted first to form a polymer (homopolymer or copolymer (e.g., random, block or the like)) prior to reaction with the polyoxyalkylene block copolymer. Conditions suitable for conducting such reactions are within the purview of those skilled in the art.

In some embodiments, in addition to a polyoxyalkylene block copolymer component, the copolymer may be combined with an epsilon-caprolactone, alone or in combination with other monomers. In one such embodiment, a polyoxyalkylene block copolymer may be reacted with a ε-caprolactone polymer containing a major amount of epsilon-caprolactone and a minor amount of at least one other copolymerizable monomer or mixture of such monomers, including the caprolactone copolymers described above in combination with a copolymerizable monomer such as glycolide.

In other embodiments, a polyoxyalkylene block copolymer may be reacted with a monomer mixture that includes a major amount of epsilon-caprolactone and a minor amount of at least one other copolymerizable monomer or mixture of such monomers in the presence of a polyhydric alcohol initiator as disclosed in U.S. Pat. No. 6,177,094. The polymerization of these monomers contemplates all of the various types of monomer addition, i.e., simultaneous, sequential, simultaneous followed by sequential, sequential followed by simultaneous, etc. Suitable monomers which can be copolymerized with epsilon-caprolactone include glycolide, lactide, p-dioxanone and trimethylene carbonate.

In embodiments, a suitable copolymer includes one possessing caprolactone units present in an amount greater than about 50% by weight, in embodiments from about 50% by weight to about 55% by weight; glycolide units present in an amount from about 5% to about 15% by weight; and a polyoxyethylene-polyoxypropylene triblock copolymer, in embodiments poloxamer 188 described above (sold under the trade name PLURONIC® F68 by BASF (Parsippany, N.J.)), in an amount from about 30% to about 45% by weight.

The compositions of the present disclosure may also possess at least one phosphorylcholine-containing polymer. Such phosphorylcholine-containing polymers are within the purview of those skilled in the art and include, for example, phosphorylcholines derived from monomers such as 2-methacryloyloxyethyl phosphorylcholine (MPC), 2-acryloyloxyethyl phosphorylcholine, and the like, and combinations thereof. Other phosphorylcholines may be utilized, including phosphorylcholines based upon monomers including, but not limited to, 2-(meth)acryloyloxyethyl-2′-(trimethylammonio)ethyl phosphate, 3-(meth)acryloyloxypropyl-2′-(trimethylammonio)ethyl phosphate, 4-(meth)acryloyloxybutyl-2′-(trimethylammonio)ethyl phosphate, 5-(meth)acryloyloxypentyl-2′-(trimethylammonio)ethyl phosphate, 6-(meth)acryloyloxyhexyl-2′-(trimethylammonio)ethyl phosphate, 2-(meth)acryloyloxyethyl-2′-(triethylammonio)ethyl phosphate, 2-(meth)acryloyloxyethyl-2′-(tripropylammonio)ethyl phosphate, 2-(meth)acryloyloxyethyl-2′-(tributylammonio)ethyl phosphate, 2-(meth)acryloyloxypropyl-2′-(trimethylammonio)ethyl phosphate, 2-(meth)acryloyloxybutyl-2′-(trimethylammonio)ethyl phosphate, 2-(meth)acryloyloxypentyl-2′-(trimethylammonio)ethyl phosphate, 2-(meth)acryloyloxyhexyl-2′-(trimethylammonio)ethyl phosphate, 2-(meth)acryloyloxyethyl-3′-(trimethylammonio)propyl phosphate, 3-(meth)acryloyloxypropyl-3′-(trimethylammonio)propyl phosphate, 4-(meth)acryloyloxybutyl-3′-(trimethylammonio)propyl phosphate, 5-(meth)acryloyloxypentyl-3′-(trimethylammonio)propyl phosphate, 6-(meth)acryloyloxyhexyl-3′-(trimethylammonio)propyl phosphate, 2-(meth)acryloyloxyethyl-4′-(trimethylammonio)butyl phosphate, 3-(meth)acryloyloxypropyl-4′-(trimethylammonio)butyl phosphate, 4-(meth)acryloyloxybutyl-4′-(trimethylammonio)butyl phosphate, 5-(meth)acryloyloxypentyl-4′-(trimethylammonio)butyl phosphate, 6-(meth)acryloyloxyhexyl-4′-(trimethylammonio)butylphosphate, and combinations thereof. As used herein, “(meth)acryl” includes both methacryl and/or acryl groups. Methods for forming phosphorylcholines from such monomers are within the purview of those skilled in the art.

In embodiments, a suitable phosphorylcholine may include a polymer obtainable by copolymerizing a radical polymerizable, in embodiments an ethylenically unsaturated, comonomer containing a group bearing a center possessing a permanent positive charge, which may be zwitterionic, and a radical polymerizable, in embodiments an ethylenically unsaturated, comonomer containing a radical polymerizable moiety and an alkyl group of 6 or more carbon atoms, which alkyl group optionally contains one or more ether oxygen atoms and optionally one or more carbon-carbon double or triple bonds, or a fluoroalkyl group which optionally contains one or more ether oxygen atoms and optionally one or more carbon-carbon double or triple bonds, or a siloxane group. In embodiments, these phosphorylcholines may be referred to herein as phosphorylcholine-containing polymers or phosphorylcholine-containing copolymers.

In other embodiments, a suitable phosphorylcholine may include a polymer obtainable by polymerizing a radical polymerizable, in embodiments ethylenically unsaturated, monomer containing a group bearing a center of permanent positive charge, which is in embodiments zwitterionic, and an alkyl group of 6 or more carbon atoms, which alkyl group optionally contains one or more ether oxygen atoms, or a fluoroalkyl group which optionally contains one or more ether oxygen atoms, or a siloxane group.

Suitable phosphorylcholines may thus include copolymers possessing residues of a radical polymerizable, in embodiments ethylenically unsaturated, comonomer containing a group bearing a center of permanent positive charge and of a radical polymerizable, in embodiments ethylenically unsaturated comonomer containing, in addition to the radical polymerizable moiety, an alkyl group of 6 or more carbon atoms which group optionally contains one or more ether oxygen atoms and optionally one or more carbon-carbon double or triple bonds, or a fluoroalkyl group which optionally contains one or more ether oxygen atoms and optionally one or more carbon-carbon double or triple bonds, or a siloxane group.

Alternatively, such a phosphorylcholine may include residues of a radical polymerizable, in embodiments ethylenically unsaturated, monomer containing a group bearing a center of permanent positive charge and an alkyl group of 6 or more carbon atoms which group optionally contains one or more ether oxygen atoms or a fluoroalkyl group which optionally contains one or more ether oxygen atoms, or a siloxane group.

In embodiments, the phosphorylcholines may be a copolymer possessing residues of a comonomer containing a physisorbable group and a comonomer containing a group bearing a center of permanent positive charge.

It is also possible that the physisorbable group may possess an alkyl or fluoroalkyl group optionally containing one or more carbon-carbon double or triple bonds. Such a group may contain one or more ether oxygen atoms, but in some embodiments it may not possess any ether oxygen atoms.

In embodiments, where the physisorbable group is an alkyl or fluoroalkyl group, optionally containing one or more ether oxygen atoms, this group may not contain any carbon-carbon double or triple bonds.

In an alternative embodiment, the present disclosure provides phosphorylcholines obtainable by copolymerizing a radical polymerizable, in embodiments ethylenically unsaturated, comonomer containing a group bearing a center of permanent positive charge, which may be zwitterionic, and a radical polymerizable, in embodiments ethylenically unsaturated, comonomer bearing a reactive group capable of covalently binding the polymer to a surface. The present disclosure also provides a polymer obtainable by polymerizing a radical polymerizable, in embodiments ethylenically unsaturated, monomer containing a group bearing a center of permanent positive charge, which may be zwitterionic, and a reactive group capable of covalently binding the phosphorylcholine-containing polymer to the surface of a substrate.

Suitable phosphorylcholines also include copolymers possessing residues of a radical polymerizable, in embodiments ethylenically unsaturated, comonomer containing a group bearing a center of permanent positive charge and a radical polymerizable, in embodiments ethylenically unsaturated, comonomer bearing a reactive group capable of covalently binding the phosphorylcholine-containing polymer to a surface of a substrate.

Alternatively, such a phosphorylcholine may include residues of a radical polymerizable, in embodiments ethylenically unsaturated, monomer containing a group bearing a center of permanent positive charge and a reactive group capable of covalently binding the phosphorylcholine-containing polymer to a surface.

In embodiments, the phosphorylcholine may be a copolymer possessing residues of a comonomer containing a group bearing a center of permanent positive charge and a comonomer containing a reactive group capable of covalently binding to the surface of a substrate.

In yet other embodiments, the present disclosure may utilize a phosphorylcholine obtainable by: (i) copolymerizing a radical polymerizable, in embodiments ethylenically unsaturated, comonomer containing a group bearing a center of permanent positive charge which may be zwitterionic, and a radical polymerizable, in embodiments ethylenically unsaturated, comonomer bearing an ionic group capable of binding to a surface by ionic interaction; or (ii) polymerizing a radical polymerizable, in embodiments ethylenically unsaturated, monomer containing a group bearing a center of permanent positive charge, which may be zwitterionic, and an ionic group capable of binding to a surface by ionic interaction.

Such a phosphorylcholine may be a copolymer possessing residues of a radical polymerizable, in embodiments ethylenically unsaturated, comonomer containing a group bearing a center of permanent positive charge, and residues of a comonomer containing an ionic group capable of binding to a surface by ionic interaction.

Alternatively such a phosphorylcholine-containing polymer may possess residues of a radical polymerizable, in embodiments ethylenically unsaturated, monomer containing a group bearing a center of permanent positive charge and an ionic group capable of binding to a surface by ionic interaction.

In embodiments, the phosphorylcholine may be a copolymer possessing residues of a comonomer containing a group bearing a center of permanent positive charge and residues of a comonomer containing an ionic group capable of binding to a surface by ionic interaction.

Optionally, in any of the above embodiments, the phosphorylcholines may also possess residues of one or more diluent and/or crosslinkable monomers.

Additional monomers and comonomers which may be used, in some embodiments, to form the phosphorylcholines of the present disclosure will now be described in more detail. As used herein, (alk)acrylate, (alk)acrylic and (alk)acrylamide mean acrylate or alkacrylate, acrylic or alkacrylic and acrylamide or alkacrylamide respectively. Unless otherwise stated, alkacrylate, alkacrylic and alkacrylamide groups may contain from about 1 to about 4 carbon atoms in the alkyl group thereof and may be methacrylate, methacrylic or methacrylamide groups. Similarly (meth)acrylate, (meth)acrylic and (meth)acrylamide shall be understood to mean acrylate or methacrylate, acrylic or methacrylic and acrylamide or methacrylamide respectively.

The monomer (or comonomer) bearing the center of permanent positive charge can either be cationic or, in embodiments, zwitterionic. In the latter case the monomer may include within its structure not only a center of permanent positive charge, but also a center of negative charge. In some embodiments the center of permanent positive charge may be provided by a quaternary nitrogen atom.

Suitable comonomers which bear a center of positive charge include those of general formula (I)

Y—B—X (I)

wherein B is a straight or branched alkylene, oxaalkylene or oligo-oxaalkylene chain optionally containing one or more fluorine atoms up to and including perfluorinated chains or, if X contains a carbon-carbon chain between B and the center of permanent positive charge, or if Y contains a terminal carbon atom bonded to B, a valence bond;

X is a group bearing a center of permanent positive charge, in embodiments a zwitterionic group; and

Y is an ethylenically unsaturated polymerizable group selected from

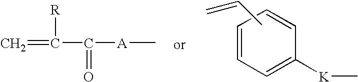

wherein R is hydrogen or a C₁-C₄ alkyl group;

A is —O— or —NR¹, where R¹ is hydrogen or a C₁-C₄ alkyl group or R¹ is —B—X where B and X are as defined above; and

K is a group such as —(CH₂)ₚOC(O)—, —(CH₂)ₚC(O)O—, —(CH₂)ₚOC(O)O—, —(CH₂)ₚNR²—, —(CH₂)ₚNR²C(O)—, —(CH₂)ₚC(O)NR², —(CH₂)ₚNR²C(O)O—, —(CH₂)ₚOC(O)NR²—, —(CH₂)ₚNR²C(O)NR²—, (in which the groups R² are the same or different) —(CH₂)ₚO—, —(CH₂)ₚSO₃—, or, optionally in combination with B, a valence bond, and p is from about 1 to about 12 and R² may be a hydrogen or a C₁-C₃ alkyl group.

The proviso on whether B may be a valence bond ensures that the center of permanent positive charge in X is not directly bonded to a heteroatom, such as an oxygen or nitrogen atom in Y.

Suitable monomers containing a group bearing a center of permanent positive charge may therefore include those of general formula (II) or (III) below:

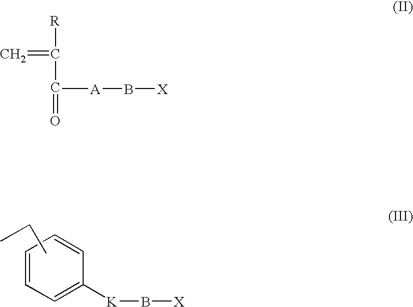

where R, A, B, K and X are as defined above with reference to formula (I).

In embodiments, in the compounds of formula (II), R may be hydrogen, methyl, or ethyl, in some cases methyl, so that (II) is an acrylic acid, methacrylic acid or ethacrylic acid derivative.

In the compounds of formula (III), K may be a valence bond and B a group, K may be a group and B a valence bond, both K and B may be groups, or K and B may together be a valence bond. In embodiments, B is a group where K is a valence bond.

Where K is a group, p may be from about 1 to about 6, in embodiments about 1, 2 or 3, and in some cases about 1. When K is a group (CH₂)ₚNR²—, —(CH₂)ₚNR²C(O)—, —(CH₂)ₚC(O)NR², —(CH₂)ₚNR²C(O)O—, —(CH₂)ₚOC(O)NR²—, or —(CH₂)ₚNR²C(O)NR²—, then R² may be hydrogen, methyl or ethyl, in some embodiments hydrogen.

In the compounds of formula (III), the vinyl group may be para to the group —K—B—X.

In embodiments, B may be:

an alkylene group of formula —(CR³₂)ₐ—, wherein the groups —(CR³₂)— are the same or different, and in each group —(CR³₂)— the groups R³ are the same or different and each group R³ may be hydrogen, fluorine or C₁₋₄ alkyl or fluoroalkyl, in embodiments hydrogen, and a is from about 1 to about 12, in embodiments from about 1 to about 6;

an oxaalkylene group such as alkoxyalkyl having from about 1 to about 6 carbon atoms in each alkyl moiety, in embodiments —CH₂O(CH₂)₄—; or

an oligo-oxaalkylene group of formula —[(CR⁴₂)_{b}O]_{c}(CR⁴₂)_{b}— where the groups —(CR⁴₂)— are the same or different and in each group —(CR⁴₂)— the groups R⁴ are the same or different and each group R⁴ is hydrogen, fluorine or C₁₋₄ alkyl or fluoroalkyl, in embodiments hydrogen, and b is from about 1 to about 6, in embodiments from about 2 to about 3, and c is from about 2 to about 11, in embodiments from about 2 to about 5; or

if X contains a carbon-carbon chain between B and the center of permanent positive charge, or if Y contains a terminal carbon atom, a valence bond.

In embodiments, suitable B groups include alkylene, oxaalkylene and oligo-oxaalkylene groups of up to about 12 carbon atoms optionally containing one or more fluorine atoms. Where the phosphorylcholine is not intended for use in a coating of a hydrophobic surface, and therefore is not intended to be bound by physiosorption to a surface, then B may be an alkylene, oxaalkylene or oligo-oxaalkylene group which does not contain any fluorine atoms.

In compounds of formula (III) it may be desirable for X and B to contain up to about 12 carbon atoms in total.

Suitable X groups containing a center of permanent positive charge, are the groups of formulae (IVA), (IVB), (IVC), (IVD), (IVE) and (IVF) as defined below.

The groups of formula (IVA) may be of the formula:

—N^{⊕}(R⁵)₃Z^{⊖} (IVA)

where the groups R⁵ are the same or different and each is hydrogen or C₁₋₄ alkyl and Z is a counter ion.

In embodiments, the groups R⁵ are all the same. In some embodiments at least one of the groups R⁵ may be methyl, so in some cases all the groups R⁵ may be methyl.

The counterion Z⁻ present in the compounds of formula (II) or (III) containing a group of formula (IVA) is such that the compounds are neutral salts. The counterion may be exchanged with ions in physiological fluids and thus the specific nature of the counterion is not critical in the present disclosure. However, physiologically acceptable counterions may be useful in some embodiments. Suitable physiologically acceptable counterions include halide anions, such as chloride or bromide ions, other inorganic anions such as sulfate, phosphate and phosphate, and organic anions such as aliphatic mono-, di- or tri-carboxylate anions containing from about 2 to about 25 carbons atoms and optionally bearing one or more hydroxyl groups, e.g., acetate, citrate and lactate.

When X is a group of formula (IVA), in some embodiments B may be a group of formula —(CR³₂)— or —(CR³₂)₂—, e.g., —(CH₂)— or —(CH₂CH₂)—.

The groups of formula (IVB) may be of the formula:

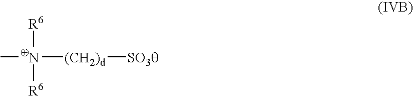

wherein R⁶ are the same or different and each may be hydrogen or C₁₋₄ alkyl, and d may be from about 2 to about 4, in embodiments from about 2 to about 3.

In embodiments, the R⁶ groups may be the same; in embodiments at least one of the R⁶ groups may be methyl, so both R⁶ groups may be methyl.

When X is a group of formula (IVB), B may be a group of formula —(CR³₂)— or —(CR³₂)₂—, e.g., —(CH₂)— or —(CH₂CH₂)—.

The groups of formula IVC may be of the formula

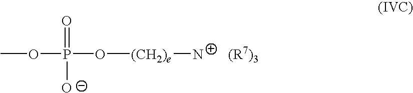

wherein the R⁷ groups may be the same or different and each may be hydrogen or C₁₋₄ alkyl, and e may be from about 1 to about 4, in embodiments from about 2 to about 3.

In embodiments the R⁷ groups may be the same; in embodiments at least one of the R⁷ groups may be methyl, so in some cases all of the R⁷ groups may be methyl.

When X is a group of formula (IVC), B may be a group of formula —(CR³₂)— or —(CR³₂)₂—, e.g., —(CH₂)— or —(CH₂CH₂)—.

The groups of formula IVD may be of the formula

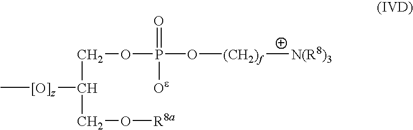

wherein the R⁸ groups are the same or different and each is hydrogen or C₁₋₄ alkyl, R^{8a} is hydrogen or a group —C(O)B¹R^{8b} where R^{8b} may be hydrogen or methyl, in embodiments methyl, B¹ is a valence bond or straight or branched alkylene, oxaalkylene or oligo-oxaalkylene group, and f may be from about 1 to about 4, in embodiments from about 1 to about 2; and

if B is other than a valence bond, z is about 1, and if B is a valence bond, z is O if X is directly bonded to an oxygen or nitrogen atom, and otherwise z is 1.

In embodiments the R⁸ groups may be the same; in embodiments at least one of the R⁸ groups may be methyl, so in some cases all of the R⁸ groups may be methyl.

In embodiments, B¹ may be:

a valence bond;

an alkylene group of formula —(CR^{3a}₂)ₐₐ—, wherein the groups —(CR^{3a}₂)— are the same or different, and in each group —(CR^{3a}₂)— the groups R^{3a} are the same or different and each group R^{3a} is hydrogen or C₁₋₄ alkyl, in embodiments hydrogen, and aa is from about 1 to about 12, in embodiments from about 1 to about 6;

an oxaalkylene group such as alkoxyalkyl having from about 1 to about 6 carbon atoms in each alkyl moiety, in embodiments —CH₂O(CH₂)₄—; or

an oligo-oxaalkylene group of formula —[(CR^{4a}₂)_{ba}O]_{ca}— where the groups —(CR^{4a}₂)— are the same or different and in each group —(CR^{4a}₂)— the groups R^{4a} are the same or different and each group R^{4a} is hydrogen or C₁₋₄ alkyl, in embodiments hydrogen, and ba is from about 1 to about 6, in embodiments from about 2 to about 3, and ca is from about 1 to about 12, in embodiments from about 1 to about 6.

Suitable B¹ groups include a valence bond and alkylene, oxaalkylene and/or oligo-oxaalkylene groups of up to about 12 carbon atoms.

In some embodiments both B and B¹ may be the same.

When X is a group of formula (IVD), B may be a group of formula —[(CR⁴₂CR⁴₂)_{c}O_{b}]CR⁴₂CR⁴₂—, e.g., —(CH₂CH₂O)_{b}(CH₂CH₂)—.

The groups of formula IVE may be of the formula

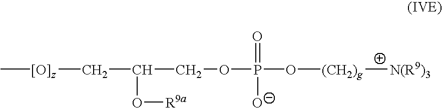

wherein the R⁹ groups may be the same or different and each is hydrogen or C₁-C₄ alkyl, R^{9a} is a hydrogen or a group —C(O)B²R^{9b}, R^{9b} is hydrogen or methyl, in embodiments methyl, B² is a valence bond or a straight or branched alkylene, oxaalkylene or oligo-oxaalkylene group, and g is from about 1 to about 4, in embodiments from about 1 to about 2; and

if B is other than a valence bond, z is 1, and if B is a valence bond z is O if X is directly bonded to an oxygen or nitrogen atom, and otherwise z is 1.

In embodiments, the R⁹ groups may be the same; in embodiments at least one of the R⁹ groups may be methyl, so in some cases all of the R⁹ groups may be methyl.

In embodiments, B² may be:

a valence bond;

an alkylene group of formula —(CR^{3b}₂)_{ab}—, wherein the groups —(CR^{3b}₂)— are the same or different, and in each group —(CR^{3b}₂)— the groups R^{3b} are the same or different and each R^{3b} group is hydrogen or C₁₋₄ alkyl, in embodiments hydrogen, and ab is from about 1 to about 12, in embodiments from about 1 to about 6;

an oxaalkylene group such as alkoxyalkyl having from about 1 to about 6 carbon atoms in each alkyl moiety, in embodiments —CH₂O(CH₂)₄—; or

an oligo-oxaalkylene group of formula —[(CR^{4b}₂)_{bb}O]_{cb}— where the groups —(CR^{4b}₂)— are the same or different and in each group —(CR^{4b}₂)— the R^{4b} groups are the same or different and each R^{4b} group is hydrogen or C₁₋₄ alkyl, in embodiments hydrogen, and bb is from about 1 to about 6, in embodiments from about 2 to about 3, and cb is from about 1 to about 12, in embodiments from about 1 to about 6.

In embodiments, the B² groups may include a valence bond and alkylene, oxalkylene and oligo-oxalkylene groups of up to about 12 carbon atoms.

In yet other embodiments, B and B² may be the same.

When X is a group of formula (IVE), B may be a group of formula —[(CR⁴₂CR⁴₂)_{b}O]_{c}CR⁴₂CR⁴₂—, e.g., —(CH₂CH₂O)_{c}(CH₂CH₂)—.

The groups of formula IVF may be of the formula

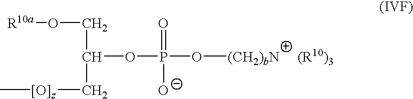

wherein the R¹⁰ groups may be the same or different and each may be hydrogen or C₁₋₄ alkyl, R^{10a} may be hydrogen or a group —C(O)B³R^{10b} where R^{10b} may be hydrogen or methyl, in embodiments methyl, B³ may be a valence bond or a straight or branched alkylene, oxaalkylene or oligo-oxaalkylene group, and h may be from about 1 to about 4, in embodiments from about 1 to about 2; and

if B is other than a valence bond z is 1, and if B is a valence bond z is O if X is directly bonded to the oxygen or nitrogen, and otherwise z is 1.

In embodiments, the R¹⁰ groups may be the same; in embodiments at least one of the R¹⁰ groups may be methyl, so in some cases all of the R¹⁰ groups may be methyl.

In embodiments, B³ may be:

a valence bond;

an alkylene group of formula —(CR^{3c}₂)_{ac}—, wherein the groups —(CR^{3c}₂)— are the same or different, and in each group —(CR^{3c}₂)— the R^{3c} groups are the same or different and each R^{3c} group is hydrogen or C₁₋₄ alkyl, in embodiments hydrogen, and ac is from about 1 to about 12, in embodiments from about 1 to about 6;

an oxaalkylene group such as alkoxyalkyl having from about 1 to about 6 carbon atoms in each alkyl moiety, in embodiments —CH₂O(CH₂)₄—; or

an oligo-oxaalkylene group of formula —[(CR^{4c}₂)_{bc}O]_{cc}— where the groups —(CR^{4c}₂)— are the same or different and in each —(CR^{4c}₂)— group the R^{4c} groups are the same or different and each R^{4c} group is hydrogen or C₁₋₄ alkyl, in embodiments hydrogen, and bc is from about 1 to about 6, in embodiments from about 2 to about 3, and cc is from about 1 to about 12, in embodiments from about 1 to about 6.

In embodiments, the B³ groups may include a valence bond and alkylene, oxalkylene and oligo-oxalkylene groups of up to about 12 carbon atoms.

In yet other embodiments, B and B³ may be the same.

When X is a group of formula (IVF), B may be a group of formula —[(CR⁴₂CR⁴₂)_{b}O]_{c}CR⁴₂CR⁴₂—, e.g., —(CH₂CH₂O)_{c}(CH₂CH₂)—.

Monomers containing such groups may be used in combination with further monomers containing groups capable of binding to a surface, to provide a phosphorylcholine suitable for use in accordance with the present disclosure. In some embodiments, it may be desirable to utilize groups of formulae (IVB)-(IVF), in some embodiments groups of formula (IVC).

Further groups bearing a center of permanent positive charge which may be utilized in forming phosphorylcholines for use in compositions of the present disclosure include those of formulae (VA), (VB) and (VC) below. These groups may also contain an alkyl or fluoroalkyl group capable of binding to a surface by physisorption. Monomers containing such a group may be suitable for use as phosphorylcholines in use with the compositions of the present disclosure, optionally without separate comonomers containing a group capable of binding to a hydrophobic surface by physisorption.

In embodiments, groups of formula (VA), (VB) and (VC) may be utilized as monomers containing both a center of permanent positive charge and an alkyl, fluoroalkyl or siloxane group capable of binding to a surface by physisorption.

The groups of formula (VA) include

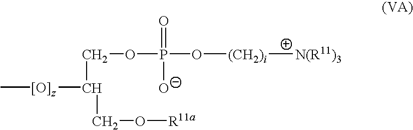

wherein the R¹¹ groups are the same or different and each is hydrogen or C₁₋₄ alkyl;

R^{11a} is either a group —[C(O)]_{vw}(CR^{11b}₂)_{ww}(SiR^{11c}₂)(OSiR^{11c}₂)ᵥᵥR^{11c} in which each group R^{11b} is the same or different and is hydrogen or alkyl of from about 1 to about 4 carbon atoms, each group R^{11c} is the same or different and is alkyl of from about 1 to about 4 carbon atoms or aralkyl, for example benzyl or phenethyl, vw is 0 or 1, ww is from about 0 to about 6 with the proviso that vw and ww are not both 0, and vv is from about 0 to about 49; or

R^{11a} is a group of formula —C(O)B⁴—R^{11d} in which R^{11d} is hydrogen or methyl, B⁴ is a valence bond or straight or branched alkylene, oxaalkylene or oligo-oxaalkalyene group optionally containing one or more fluorine atoms, and containing from about 6 to about 24 carbon atoms, in embodiments from about 6 to about 18 carbon atoms; i is from about 1 to about 4; and

if B is other than a valence bond Z is 1, and if B is a valence bond Z is 0 if X is directly bonded to an oxygen or nitrogen atom, and otherwise Z is 1.

In embodiments, the R¹¹ groups may be the same; in embodiments at least one of the R¹¹ groups may be methyl, so in some cases all of the R¹¹ groups may be methyl.

Where R^{11a} is a siloxane group as defined above, each group (CR^{11b}₂) may be the same or different, in embodiments the same, and each group CR^{11b}₂ may be hydrogen. In embodiments ww may be from about 2 to about 4, and may be about 3 when vw is 0, or about 2 when vw is 1. Each group (SiR^{11c}₂) may be the same or different, in embodiments the same, and in some embodiments each R^{11c} may be methyl. In some embodiments vv may be from about 4 to about 29.

In embodiments, R^{11a} may be a group —C(O)B⁴R^{11d} as defined above. In such a case, B⁴ may be:

a valence bond;

an alkylene group of formula —(CR^{3d}₂)_{ad}—, wherein the groups —(CR^{3d}₂)— are the same or different, and in each group —(CR^{3d}₂)— the groups R^{3d}₂ are the same or different and each group R^{3d}₂ is hydrogen, fluorine or C₁₋₄ alkyl or fluoroalkyl, in embodiments hydrogen or fluorine, and ad may be from about 1 to about 24, in embodiments from about 6 to about 18;

an oxaalkylene group such as alkoxyalkyl having from about 1 to about 6 carbon atoms and optionally one or more fluorine atoms in each alkyl moiety, or

an oligo-oxalkylene group of formula —[(CR^{4d}₂)_{bd}O]_{cd}— where the groups —(CR^{4d}₂)— are the same or different and in each group —(CR^{4d}₂)— the groups R^{4d} are the same or different and each group R^{4d} is hydrogen, fluorine or C₁₋₄ alkyl or fluoroalkyl, in embodiments hydrogen or fluorine, and bd is from about 2 to about 6, in embodiments from about 3 to about 4, and cd is from about 1 to about 12, in embodiments from about 1 to about 6.

When B⁴ is a group —[(CR^{4d}₂)_{bd}O]_{cd}— wherein all the groups R^{4d} are hydrogen and bd is 2, the residues of the monomer of formula (VA) may not be able to form strong secondary valence interactions with hydrophobic surfaces. While residues of such monomers may be included in the phosphorylcholines utilized with the compositions of the present disclosure, it may be desirable to include residues of monomers which are capable of forming strong secondary valence interactions if such interactions are to bind a polymer to a surface.

Monomers which have groups containing oligo(higher alkylene) oxide moieties can be used to provide strong secondary valence interactions. Monomers which contain oligo alkylene oxide moieties can also be utilized in which at least about 50, in embodiments about 70, in other embodiments about 90 mol % of individual alkylene oxide units contain 3 or more carbon atoms. Thus, in embodiments, a mixed oligo(ethylene oxide/propylene oxide) side chain could be used if there are more propylene oxide units than ethylene oxide units.

When B⁴ is a group —[(CR^{4d}₂)_{bd}O]_{cd}— then bd may be about 2 in only about 50, in embodiments about 70, in other embodiments about 90 mole % or less of the residues —[(CR^{4d}₂)_{bd}O]—.

When the group —B⁴—R^{11a} is a group capable of forming strong secondary valence interactions with a surface, then monomers containing a group (VA) may be particularly suitable for use as monomers containing a group bearing a center of permanent positive charge and an alkyl or fluoroalkyl group optionally containing one or more ether oxygen atoms. In such a case —B⁴—R^{11a} may be an alkyl group optionally containing one or more ether oxygen atoms and in embodiments 6 or more carbon atoms or a fluoroalkyl group optionally containing one or more ether oxygen atoms and in embodiments 6 or more carbon atoms.

In one embodiment B and B⁴ may be the same.

The groups of formula (VB) include

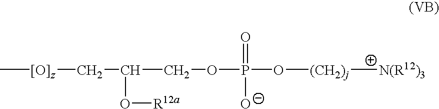

wherein the groups R¹² are the same or different and each is hydrogen or C₁-C₄ alkyl,

R^{12a} is either a group —[C(O)]ₜᵤ(CR^{12b}₂)ᵤᵤ(SiR^{12c}₂)(OSiR^{12c}₂)ₜₜR^{12c} in which each group R^{12b} is the same or different and is hydrogen or alkyl of from about 1 to about 4 carbon atoms, each group R^{12c} is the same or different and is alkyl of from about 1 to about 4 carbon atoms or aralkyl, for example benzyl or phenethyl, tu is 0 or 1, uu is from about 0 to about 6, with the proviso that tu and uu are not both 0, and tt is from about 0 to about 49; or

R^{12a} is a group forumla —C(O)B⁵—R^{12d}, in which R^{12d} is hydrogen or methyl, B⁵ is a valence bond or a straight or branched alkylene, oxaalkylene or oligo-oxaalkylene group optionally containing one or more fluorine atoms and from about 6 to about 24 carbon atoms, in embodiments from about 6 to about 18 carbons atoms,

j is from about 1 to about 4, in embodiments from about 1 to about 2; and

if B is other than a valence bond Z is 1, and if B is a valence bond Z is 0 if X is directly bonded to an oxygen or nitrogen atom, and otherwise Z is 1.

In embodiments, the R¹² groups may be the same; in embodiments at least one of the R¹² groups may be methyl, so in some cases all of the R¹² groups may be methyl.

Where R^{12a} is a siloxane group as defined above, each group (CR^{12b}₂) may be the same or different, in embodiments the same, and in some embodiments each group R^{12b} may be hydrogen. In embodiments, uu may be from about 2 to about 4, and may be 3 when tu is 0, or 2 when tu is 1. Each group (Si R^{12c}₂) may be the same or different, in embodiments the same, and each R¹² may be methyl. In embodiments, tt may be from about 4 to about 29.

In embodiments, R^{12a} may be a group —C(O)B⁴R^{12d} as defined above. In such a case, B⁵ may be:

a valence bond;

an alkylene group of formula —(CR^{3c}₂)_{ce}—, wherein the groups —(CR^{3c}₂)— are the same or different, and in each group —(CR^{3c}₂)— the groups R^{3c} are the same of different and each group R^{3c} is hydrogen, fluorine or C₁₋₄ alkyl, or fluoroalkyl, in embodiments hydrogen or fluorine, and ce may be from about 1 to about 24, in embodiments from about 6 to about 18;

an oxaalkylene group such as alkoxyalkyl having from about 1 to about 6 carbon atoms and optionally one or more fluorine atoms in each alkyl moiety; or

an oligo-oxaalkylene group of formula —[(CR^{4c}₂)_{be}O]_{ce}— where the groups —(CR^{4c}₂)— are the same or different and in each group —(CR^{4c}₂)— the groups R^{4c} are the same or different and each group R^{4c} is hydrogen, fluorine or C₁₋₄ alkyl or fluoroalkyl, in embodiments hydrogen or fluorine, and be is from about 2 to about 6, in embodiments from about 3 to about 4, and ce is from about 1 to about 12, in embodiments from about 1 to about 6.

When B⁵ is a group —[(CR^{4c}₂)_{be}O]_{ce}— wherein all the groups R^{4c} are hydrogen and be is 2 in all the groups [(CR^{4c}₂)_{be}O], the residues of the monomer of formula (VB) may not be able to form strong secondary valence interactions with hydrophobic surfaces. While residues of such monomers may be included in the phosphorylcholines utilized in compositions of the present disclosure, it may be necessary to include residues of monomers which are capable of forming such strong secondary valence interactions if such interactions are to bind a polymer to a surface.

Monomers which have groups containing oligo(higher alkylene) oxide moieties can be used to provide strong secondary valence interactions. Monomers which contain oligo alkylene oxide moieties can also be utilized in which at least about 50, in embodiments about 70, in other embodiments about 90 mol % of individual alkylene oxide units contain 3 or more carbon atoms. Thus, in embodiments, a mixed oligo(ethylene oxide/propylene oxide) side chain could be used if there are more propylene oxide units than ethylene oxide units.

When B⁵ is a group —[(CR^{4c}₂)_{be}O]_{ce}— then be may be 2 in only about 50, in embodiments about 70, in other embodiments about 90 mole % or less of the residues —[(CR^{4c}₂)_{be}O]—.

When the group —B⁵—R^{12a} is a group capable of forming strong secondary valence interactions with a surface, then monomers containing a group (VB) may be particularly suitable for use as monomers containing a group bearing a center of permanent positive charge and an alkyl or fluoroalkyl group optionally containing one or more ether oxygen atoms. In such a case —B⁵—R^{12a} may be an alkyl group optionally containing one or more ether oxygen atoms and, in embodiments, about 6 or more carbon atoms, or a fluoroalkyl group optionally containing one or more ether oxygen atoms and about 6 or more carbon atoms.

In one embodiment B and B⁵ may be the same.

The groups of formula (VC) include

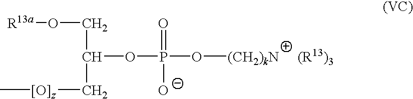

wherein the groups R¹³ are the same or different and each is hydrogen or C₁₋₄ alkyl,

R^{13a} is either a group —[C(O)]ᵣₛCR^{13b}₂)ₛₛ(Si R^{13c}₂)(OSiR^{13c}₂)ᵣᵣR^{13c} in which each group R^{13b} is the same or different and is hydrogen or alkyl of from about 1 to about 4 carbon atoms, each group R^{13c} is the same or different and is alkyl of from about 1 to about 4 carbon atoms or aralkyl, for example benzyl or phenethyl, rs is 0 or 1, ss is from about 0 to about 6, with the proviso that rs and ss are not both 0, and rr is from about 0 to about 49; or

R^{13a} is a group of formula —C(O)B⁶—R^{13d}, in which R^{13d} is hydrogen or methyl, B⁶ is a valence bond or a straight or branched alkylene, oxaalkylene or oligo-oxaalkylene group optionally containing one or more fluorine atoms and from about 6 to about 24, in embodiments from about 6 to about 18, carbon atoms,

and k is from about 1 to about 4, in embodiments from about 1 to about 2; and

if B is other than a valence bond Z is 1, and if B is a valence bond Z is 0 if X is directly bonded to an oxygen or nitrogen atom, and otherwise Z is 1.

In embodiments, the R¹³ groups may be the same; in embodiments at least one of the R¹³ groups may be methyl, so in some cases all of the R¹³ groups may be methyl.

Where R^{13a} is a siloxane group as defined above, each group (CR^{13b}₂) may be the same or different, in embodiments the same and in embodiments each group R^{13b} may be a hydrogen. In embodiments ss may be from about 2 to about 4, and may be 3 when rs is 0, or 2 when rs is 1. Each group (SiR^{13c}₂) may be the same or different, in embodiments the same, and each group R^{13c} may be methyl. In embodiments rr may be from about 4 to about 29.

In embodiments, R^{13a} may be a group —C(O)B⁶R^{13d} as defined above. In such a case, B⁶ may be:

a valence bond;

an alkylene group of formula —(CR^{3f}₂)_{af}—, wherein the groups —(CR^{3f}₂)— are the same or different, and in each group —(CR^{3f}₂)— the groups R^{3f} are the same or different and each R^{3f} group is hydrogen, fluorine or C₁₋₄ alkyl or fluoroalkyl, in embodiments hydrogen or fluorine, and of is from about 1 to about 24, in embodiments from about 6 to about 18;

an oxaalkylene group such as alkoxyalkyl having from about 1 to about 6 carbon atoms and optionally one or more fluorine atoms in each alkyl moiety; or

an oligo-oxaalkylene group of formula —[(CR^{4f}₂)_{bf}O]_{cf}— where the groups —(CR^{4f}₂)— are the same or different and in each group —(CR^{4f}₂)— the groups R^{4f} are the same or different and each R^{4f} group is hydrogen, fluorine or C₁₋₄ alkyl or fluoroalkyl, in embodiments hydrogen or fluorine, and bf is from about 2 to about 6, in embodiments from about 3 to about 4, and cf is from about 1 to about 12, in embodiments from about 1 to about 6.

When B⁶ is a group —[(CR^{4f}₂)_{bf}O]_{cf}— wherein all the groups R^{4f} are hydrogen and in all the groups [(CR^{4f}₂)_{bf}O] bf is 2, the residues of the monomer of formula (VC) may not be able to form strong secondary valence interactions with hydrophobic surfaces. While residues of such monomers may be included in the phosphorylcholines utilized to form compositions of the present disclosure, it may be necessary to include residues of monomers which are capable of forming such strong secondary valence interactions if such interactions are to bind a polymer to a surface.

Monomers which have groups containing oligo(higher alkylene) oxide moieties can be used to provide strong secondary valence interactions. Monomers which contain oligo alkylene oxide moieties can also be utilized in which at least about 50, in embodiments about 70, in other embodiments about 90 mol % of individual alkylene oxide units contain 3 or more carbon atoms. Thus, in embodiments, a mixed oligo(ethylene oxide/propylene oxide) side chain could be used if there are more propylene oxide units than ethylene oxide units.

When B⁶ is a group —[(CR^{4f}₂)_{bf}O]_{cf}— then bf may be 2 in only about 50, in embodiments about 70, in other embodiments about 90 mol % or less of the residues —[(CR^{4f}₂)_{bf}O]—.

When the group —B⁶—R^{13a} is a group capable of forming strong secondary valence interactions with a surface, then monomers containing a group (VC) may be particularly suitable for use as monomers containing a group bearing a center of permanent positive charge and an alkyl or fluoroalkyl group optionally containing one or more ether oxygen atoms. In such a case —B⁶—R^{13a} may be an alkyl group optionally containing one or more ether oxygen atoms and, in embodiments, about 6 or more carbon atoms, or a fluoroalkyl group optionally containing one or more ether oxygen atoms and in embodiments about 6 or more carbon atoms.

In one embodiment B and B⁶ may be the same.

Particular examples of suitable monomers bearing a group containing a center of permanent positive charge are 2 (methacryloyloxy)ethyl-2′(trimethylammonium) ethyl phosphate inner salt and 1[4(4′-vinylbenzyloxy)butane]-2″(trimethylammonium)ethyl phosphate inner salt.

Monomers bearing a group containing a center of permanent positive charge, such as those of formula (II) and (III) may be prepared by conventional techniques using known reactions, for example using a suitable substituted alkyl (alk)acrylate or suitable substituted styrene as precursor. Examples of suitable substituted alkyl (alk)acrylates include dimethylaminoethyl(meth)acrylate and 2-hydroxyethyl(meth)acrylate.

Monomers of formula (II) or (III) containing a group of formula (IVA) may be prepared by methods within the purview of those skilled in the art. Similarly, monomers containing a group of formula (IVB) or (IVC) may be prepared by methods within the purview of those skilled in the art.

Monomers of formula (II) or (III) containing a group of formula (IVD) in which R^{8a} is —C(O)B¹R^{8b} may be prepared by selective acylation of glycerophosphoryl phosphorylcholine or analogues thereof at the primary hydroxyl group with an activated acid derivative such as an acid anhydride O(C(O)B¹R^{8b})₂ or an acid halide R^{8b}B¹ COHal where B¹ and R^{8b} are as defined above and Hal is halogen, followed by acylyzation of the secondary hydroxyl group with an appropriate acylating agent, for example methacryloyl chloride. Purification, for example by column chromatography on a suitable support, may be performed after each acylation or after the second acylation only. Suitable activated acid derivatives include acid anhydrides, acid halides, reactive esters and imidazolides. The acylations may be performed in a suitable anhydrous, aprotic solvent, for example N,N-dimethylformamide, optionally in the presence of a suitable non-nucleophilic base, for example triethylamine.

Alternatively, the primary alcohol group in glycerophosphoryl choline or an analogue thereof may be blocked by reaction with a suitable protecting group reagent, for example t-butyldimethylsilyl chloride, under standard conditions and the secondary hydroxy group then treated with an acylating agent such as methacryloyl chloride. The t-butyldimethylsilyl protecting group may be removed by treatment with a dilute organic or mineral acid, for example p-toluene sulfonic acid, hydrochloric acid or with tetra-butylammonium fluoride. The deblocked primary hydroxyl group may then be treated with an activated acid derivative such as an acid anhydride O(C(O)B¹R^{8b})₂ or an acid halide R^{8b}B¹COHal where B¹ and R^{8b} are as defined above, and Hal is halogen.

Analogues of glycerophosphorylcholine (compounds of formula (II) or (III) containing a group (IVD) where R^{8a} is hydrogen) may be prepared by reaction of phosphorus oxychloride with a bromoalcohol in an inert aprotic solvent, such as dichloromethane, to give a bromoalkylphosphorodichloridate. The dichloro derivative thus produced may then be treated with an appropriately protected glycerol derivative, for example 2,2-dimethyl 1,3-dioxolane-4-methanol, in the presence of a base, for example triethylamine, followed by acid hydrolysis to give a bromoalkylphosphoroglycerol derivative. This may then be treated with an amine NR⁸₃, where R⁸ is as defined above, for example trimethylamine, to generate the glycerophosphorylcholine analogue.

Monomers of formula (II) or (III) containing a group of formula (IVE) in which R^{9a} is —C(O)B²R^{9b} may be prepared by the selective acylation of glycerophosphorylcholine or an analogue thereof at the primary hydroxyl group with for example, methacryloyl chloride followed by reaction at the secondary hydroxyl group using an activated acid derivative, such as an acid halide O(C(O)B²R^{9b})₂ or an acid halide R^{9b}B²COHal, where B² and R^{9b} are as defined above and Hal is halogen. The intermediates and final products may be purified, as necessary using column chromatography. Optionally, protecting group strategy, similar to that outlined above in relation to production of monomers containing a group of formula (IVD) may be employed.

Monomers of formula (II) or (III) containing a group of formula (IVF) may be prepared in an analogous manner to monomers containing groups of formula (IVD) or (IVE).

Monomers of formula (II) or (III) containing a group of formula (VA), (VB) or (VC) may be prepared by direct analogy with methods described for monomers containing groups of formula (IVD), (IVE) and (IVF) respectively.

For the above phosphorylcholines useful in forming the compositions of the present disclosure, where the group bearing a center of permanent positive charge and group capable of stably binding the polymer to a surface are not present in the residue of the same monomer, the phosphorylcholine may include residues of a comonomer containing a group capable of stably binding the phosphorylcholine to a surface as well as the residues of the comonomer containing a group bearing a center of permanent positive charge. Optionally, where the monomer containing a group bearing a center of permanent positive charge also contains a group capable of stably binding the phosphorylcholine to a surface, further groups capable of stably binding to a surface may be provided by additional comonomer residues containing a group capable of binding the phosphorylcholine to a surface.

As has already been mentioned, the nature of the group capable of binding to a surface, and therefore the nature of the comonomers containing such groups, will depend upon the nature of the surface which is to be coated with a composition of the present disclosure possessing such a phosphorylcholine.

It will be appreciated that in some circumstances it may be desirable to use a combination of different comonomers containing groups capable of binding to a surface. In embodiments, a comonomer of type a), b) and/or c) as defined below or a combination of such comonomers may be used, in some cases only one of comonomer types a), b) and c) may be used.

Type a) comonomers include those containing an alkyl, fluoroalkyl or siloxane group. Comonomers containing an alkyl, fluoroalkyl or siloxane group, which are suitable for providing binding to a hydrophobic surface include comonomers containing an alkyl group of 6 or more carbon atoms which group optionally contains one or more ether oxygen atoms and optionally one or more carbon-carbon double or triple bonds or a fluoroalkyl group in embodiments of 6 or more carbon atoms, which group optionally contains one or more ether oxygen atoms and optionally one or more carbon-carbon double or triple bonds, or containing a siloxane group, containing up to about 50 silicon atoms, in embodiments in a linear chain.

In embodiments, the alkyl or fluoroalkyl groups contain up to about 24 carbon atoms, for instance up to about 18 carbon atoms, or containing a siloxane group, containing up to about 50 silicon, in embodiments in a linear chain. Suitable comonomers containing an alkyl, fluoroalkyl or siloxane group include those of general formula VI

Y¹-Q (VI)

where Y¹ is an ethylenically unsaturated polymerizable group selected from

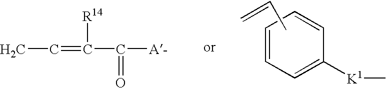

where R¹⁴ is hydrogen or C₁-C₄ alkyl, A′ is —O— or —NR¹⁵— where R¹⁵ is hydrogen or a C₁-C₄ alkyl group or R¹⁵ is a group Q;

K¹ is a group —(CH₂)₁OC(O)—, —(CH)₁C(O)O—, —(CH₂)₁OC(O)O—, —(CH₂)₁NR¹⁶—, —(CH₂)₁NR¹⁶C(O)—, —(CH₂)₁C(O)NR¹⁶—, —(CH₂)₁NR¹⁶C(O)O—, —(CH₂)₁OC(O)NR¹⁶—, —(CH₂)₁NR¹⁶C(O)NR¹⁶—(in which the groups R¹⁶ are the same or different), —(CH₂)₁O—, —(CH₂)₁SO₃—, a valence bond and 1 is from about 1 to about 12 and R¹⁶ is hydrogen or a C₁-C₄ alkyl group; and

Q is (a) a straight or branched alkyl, alkoxyalkyl or (oligo-alkoxy)alkyl chain containing about 6 or more, in embodiments from about 6 to about 24, carbon atoms unsubstituted or substituted by one or more fluorine atoms and optionally containing one or more carbon-carbon double or triple bonds; or

(b) a siloxane group —(CR^{16a}₂)_{qq}(OSiR^{16b}₂)ₚₚR^{16b} in which each group R^{16a} is the same or different and is hydrogen or alkyl of from about 1 to about 4 carbon atoms or aralkyl, for example benzyl or phenethyl, each group R^{16b} is alkyl of from about 1 to about 4 carbon atoms, qq is from about 1 to about 6 and pp is from about 0 to about 49.

In embodiments, suitable comonomers of formula (VI) bearing a group Q include those of formula (VII) and (VIII):

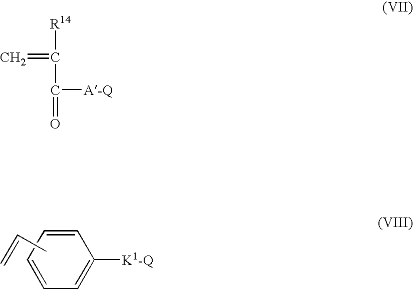

wherein R¹⁴, A′, K¹ and Q are as defined in relation to formula (VI) above.

In embodiments, in the compounds of formula (VII), R¹⁴ is hydrogen methyl or ethyl, in some cases methyl, so that the compound of formula (VII) is an acrylic acid, methacrylic acid or ethacrylic acid derivative.

In the compounds of formula (VIII) K¹ may, for instance, be a valence bond. Where K¹ is a group, then 1 may be from about 1 to about 6, in embodiments about 1, 2 or 3, and in some cases 1 may be 1. When K¹ is a group —(CH₂)₁NR¹⁶—, —(CH₂)₁OC(O)NR¹⁶—, —(CH₂)₁NR¹⁶C(O)O—, —(CH₂)₁NR¹⁶C(O)—, —(CH₂)₁C(O)NR¹⁶— or —(CH₂)₁NR¹⁶C(O)NR¹⁶— then NR¹⁶ may be hydrogen, methyl or ethyl, in embodiments hydrogen.

In the compounds of formula (VIII), the vinyl group may be para to the group —K¹-Q.

Q may be an alkyl or fluoroalkyl group optionally containing one or more ether oxygen atoms and optionally one or more carbon-carbon double or triple bonds. In embodiments, Q may be:

an alkyl group of formula —(CR¹⁷₂)ₘCR¹⁷₃ wherein the groups —(CR¹⁷₂)— are the same or different, and in each group —(CR¹⁷₂)— the groups R¹⁷ are the same or different and each group R¹⁷₂ is hydrogen, fluorine or C₁₋₄ alkyl or fluoroalkyl, and m is from about 5 to about 23 if Q contains no fluorine atoms or from about 1 to about 23, in embodiments from about 5 to about 23, if Q contains one or more fluorine atoms;

an alkoxyalkyl having from about 1 to about 12 carbon atoms in each alkyl moiety; unsubstituted or substituted by one or more fluorine atoms; or

an (oligo-alkoxyl) alkyl group of formula —[(CR¹⁸₂)ₙO]ₒ(CR¹⁸₂)ₙR¹⁸ where the groups —(CR¹⁸₂)— are the same or different and in each group —(CR¹⁸₂)— the groups R¹⁸ are the same or different and each group R¹⁸ is hydrogen, fluorine or C₁₋₄ alkyl or fluoroalkyl and n is from about 2 to about 6, in embodiments from about 3 to about 4, and o is from about 1 to about 12.

When Q is a group —[(CR¹⁸₂)ₙO]ₒ(CR¹⁸₂)ₙR¹⁸ wherein all the groups R¹⁸ are hydrogen and in all the groups —[(CR¹⁸₂)ₙO]ₒ— n is 2, the group of formula Q may not be able to form strong secondary valence interactions with hydrophobic surfaces. While residues of monomers containing such a group may be included in phosphorylcholines utilized to form compositions of the present disclosure, it may be desirable to include residues of monomers which are capable of forming such strong secondary valence interactions if such interactions are to bind a polymer to a surface. Monomers which have groups containing oligo(higher alkylene) oxide moieties can be used to provide monomers which contain oligo alkylene oxide moieties in which at least about 50 mol % of individual alkylene oxide units contain about 3 or more carbons atoms. Thus, for instance a mixed oligo(ethylene oxide/propylene oxide) side chain could be used provided that there are more propylene oxide units than ethylene oxide units.

Where Q is an (oligo-alkoxy)-alkyl group containing residues —[(CR¹⁸₂)ₙO]— wherein n is 2, then n may be 2 in no more than 50 mol % of the residues —[(CR¹⁸₂)ₙO]—.

Alternatively, Q may be a group in which one or more of the alkyl or alkylene moieties in such an alkyl, alkoxyalkyl or (oligoalkoxy) alkyl group is replaced by a corresponding alkenyl, alkynyl, alkenylene or alkynylene moiety.

Suitable Q groups include alkyl, alkoxyalkyl and (oligo-alkoxy)alkyl groups optionally containing one or more carbon-carbon double or triple bonds of 8 or more, in embodiments 10 or more, in other embodiments 12 or more, for instance 14 or more, such as 16 or more carbon atoms. Such groups may contain one or more fluorine atoms and be therefore fluoroalkyl derivatives. In some embodiments, however, such groups may not contain any fluorine atoms.

Suitable groups include straight chain alkyl or fluoroalkyl groups optionally containing one or more carbon-carbon double or triple bonds.

Where Q is a siloxane group, each group —(CR^{16a}₂)— may be the same or different, in embodiments the same, and in some cases each R^{16a} group may be hydrogen. In embodiments qq may be from 2 about to about 4, in some embodiments about 3. Each group —(SiR^{16b}₂)— may be the same or different, in embodiments the same, and in some embodiments each R^{16b} group may be methyl. In embodiments pp may be from about 4 to about 29. Suitable comonomers where Q is a siloxane group include those of formula (VII).

In one specific embodiment the group Q does not contain any ethylenic unsaturation, i.e. any carbon-carbon double or triple bonds.

Particular examples of comonomers containing an alkyl, fluoroalkyl or siloxane group include: n-dodecyl methacrylate, octadecyl methacrylate, hexadecyl methacrylate, 1H,1H,2H,2H-heptadecafluorodecyl methacrylate, p-octyl styrene, p-dodecyl styrene and monomethacryloxypropyl terminated siloxanes. n-Dodecyl methacrylate may be suitable in some embodiments.

Comonomers containing a physisorbable alkyl or fluoroalkyl, which does not contain a carbon-carbon double or triple bond, or a siloxane group such as those of formulae (VII) and (VIII) are commercially available or may be prepared by conventional techniques using known reactions.

In other embodiments, the Q group may contain ethylene unsaturation, i.e. one or more carbon-carbon double or triple bonds. Such comonomers may, for example, contain a vinylic, divinylic, acetylenic or diacetylenic moiety. Comonomers containing acetylenic rather than vinylic unsaturation may be suitable, especially those containing a single acetylenic group.

Comonomers which contain such an ethylenic unsaturated group may be capable of providing crosslinking between linear polymer claims once the polymer is coated onto a substrate, as well as binding to the substrate by physisorption. Such crosslinking may improve the stability of the coating and may be formed by irradiation, for example with UV- or gamma-radiation. The crosslinking of such groups may be employed either alone or in addition to the use of a comonomer containing a reactive group as a crosslinkable comonomer as described below.

Suitable crosslinkable comonomers capable of binding to a substrate by physisorption include those of formulae

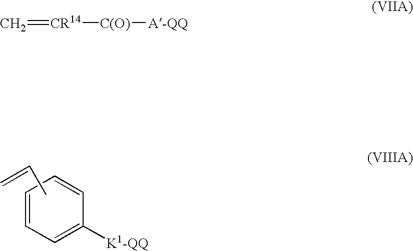

in which R¹⁴, A′ and K¹ are as hereinbefore defined and QQ is an alkynyl group containing 6 or more carbon atoms and one or two, in embodiments one, carbon-carbon triple bonds, provided that the acetylenic moieties are not directly bonded to A′ or K¹.

The present disclosure provides, as a further feature, comonomers of formula (VIIA) and (VIIIA).

Amongst such comonomers it may be desirable that QQ is a group containing from about 6 to about 24 carbon atoms, in embodiments about 8 or more, in some cases about 10 or more, about 12 or more, about 14 or more, or even about 16 or more carbon atoms in some embodiments.

It may also be desirable that the group QQ does not contain a terminal acetylenic moiety, i.e. a group —C≡CH.

In some embodiments the QQ group may be 7-dodecynyl and a specific example of a compound of formula (VIIA) containing such a group is dodec-7-yn-1-ol methacrylate.

The compounds of formula (VIIA) and (VIIIA) and other comonomers of formula (VII) and (VIII) containing an ethylenically unsaturated physisorbable group Q, may be prepared by any methods within the purview of those skilled in the art.

In other embodiments, comonomers possessing a reactive group may be utilized to form phosphorylcholines suitable for use in compositions of the present disclosure. Suitable comonomers, which may be useful for providing binding to a hydrophilic surface having functional groups, may contain a reactive group capable of covalently binding to a surface and are of general formula

Y²-Q¹ (IX)

where Y² is an ethylenically unsaturated polymerizable group selected from

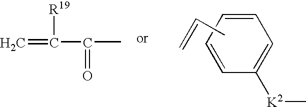

where R¹⁹ is hydrogen or C₁-C₄ alkyl, K² is a group —(CH₂)_{q}OC(O)—, —(CH)_{q}C(O)O—, —(CH₂)_{q}OC(O)O—, —(CH₂)_{q}NR²⁰—, —(CH₂)_{q}NR²⁰C(O)—, —(CH₂)_{q}C(O)NR²⁰—, —(CH₂)_{q}NR²⁰C(O)O—, —(CH₂)_{q}C(O)NR²⁰—, —(CH₂)_{q}NR²OC(O)NR²⁰— (in which the groups R²⁰ are the same or different), —(CH₂)_{q}O—, or —(CH₂)_{q}SO₃—, or a valence bond and q is from about 1 to about 12 and R²⁰ is hydrogen or a C₁-C₄ alkyl group; and

Q¹ is a reactive group capable of reacting to provide covalent binding to a surface.

Suitable comonomers of formula (IX) bearing a reactive group Q¹ include those of formula (X) and (XI) defined below.

Compounds of formula (X) include

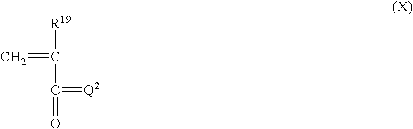

wherein R¹⁹ is as defined with reference to formula (IX), and Q² is a reactive group. In embodiments, R¹⁹ may be hydrogen, methyl or ethyl, in some cases methyl, so that the compound of formula (X) may be an acrylic acid, methacrylic acid or ethacrylic acid derivative.

In some embodiments Q² may be hydrogen, —OH or a group of the formula:

-T—B⁷-Q³

where T is —O—, or —NR²¹— where R²¹ may be hydrogen, C₁-C₄ alkyl or a group —B⁷-Q³;

B⁷ is a valence bond or, in embodiments, a straight or branched alkylene, oxaalkylene or oligo-oxaalkylene chain; and

Q³ is a reactive group capable of reacting to provide covalent binding to a surface such as an aldehyde group or a silane or siloxane group containing one or more reactive substituents such as halogen, for example chlorine, or alkoxy, generally containing from about 1 to about 4 carbon atoms, for example methoxy or ethoxy, or, in embodiments Q³ may be a hydroxyl, amino, carboxyl, epoxy, —CHOHCH₂Hal, (in which Hal is a halogen atom such as chlorine, bromine or iodine) succinimide, tosylate such as 2(N-methylpyridinium) tosylate, triflate, imidazole carbonyl-amino, or an optionally substituted triazine group.

In embodiments, B⁷ may be:

an alkylene group of formula —(CR²²₂)ᵣ—, wherein the groups —(CR²²₂)— are the same or different, and in each group —(CR²²₂)— the groups R²² groups are the same or different and each group R²² is hydrogen or C₁₋₄ alkyl, in embodiments hydrogen, and r is from about 1 to about 12, in embodiments from about 1 to about 6;

an oxaalkylene group such as alkoxyalkyl having from about 1 to about 6 carbon atoms in each alkyl moiety; or

an oligo-oxaalkylene group of formula —[(CR²³₂)ₛO]₁(CR²³₂)ₛ— where the groups —(CR²³₂)— are the same or different and in each group —(CR²³₂)— the R²³ groups are the same or different and each R²³ may be hydrogen or C₁₋₄ alkyl, in embodiments hydrogen, and s is from about 1 to about 6, in embodiments from about 2 to about 3, and t is from about 1 to about 11, in embodiments from about 1 to about 5.

Suitable B⁷ groups include alkylene, oxaalkylene and oligo-oxaalkylene groups of up to about 12 carbon atoms.

Where Q³ is a silane or siloxy group, B⁷ may be an alkylene group of from about 1 to about 6, in embodiments from about 2 to about 4, in some cases about 3 carbon atoms.

Particular examples of the group B⁷ include —CH₂—, —CH₂CH₂— and —(CH₂)₆—.

The compounds of formula (XI) include:

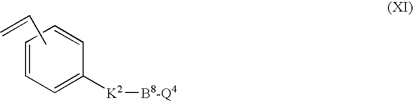

wherein K² is as defined in relation to formula (IX), B⁸ is a straight of branched alkylene, oxaalkylene or oligo-oxaalkylene chain, and Q⁴ is a reactive group capable of reacting to provide covalent binding to a surface, for example an aldehyde group or a silane or siloxane group containing one or more reactive substituents such as halogen, for example chlorine, or alkoxy generally containing from about 1 to about 4 carbon atoms, for example methoxy or ethoxy, or, in some embodiments Q⁴ is a hydroxyl, amino, carboxyl, epoxy,

—CHOHCH₂Hal, (in which Hal is a halogen atom such as chlorine, bromine or iodine) succinimide, tosylate, triflate, imidazole carbonyl-amino or optionally substituted triazine group.

In the compounds of formula (XI), the vinyl group may be para to the group —K²—B⁸-Q⁴.

K² may, for instance, be a valence bond. Where K² is a group then q may be from about 1 to about 6, in embodiments about 1, 2 or 3, in some cases q is about 1. When K² is a group —(CH₂)_{q}NR²⁰—, —(CH₂)_{q}OC(O)NR²⁰—, —(CH₂)_{q}NR²⁰C(O)O—, —(CH₂)_{q}NR²⁰C(O)—, —(CH₂)_{q}C(O)NR²⁰— or —(CH₂)_{q}NR²⁰— then R²⁰ may be hydrogen, methyl or ethyl, in some cases hydrogen.

In some embodiments, B⁸ may be:

an alkylene group of formula —(CR²⁴₂)ᵤ—, wherein the groups —(CR²⁴₂)— are the same or different, and in each group —(CR²⁴₂)— the groups R²⁴ are the same of different and each group R²⁴ may be hydrogen or C₁₋₄ alkyl, in embodiments hydrogen, and u is from about 1 to about 12, in embodiments from about 1 to about 6;

an oxaalkylene group such as alkoxyalkyl having from about 1 to about 6 carbon atoms in each alkyl moiety; or

an oligo-oxaalkylene group of formula —(CR²⁵₂)ᵥO]_{w}(CR²⁵)ᵥ— where the groups —(CR²⁵₂)— are the same or different and in each group —[(CR²⁵₂)— the groups R²⁵ are the same or different and each group R²⁵ is hydrogen or C₁₋₄ alkyl, in embodiments hydrogen, and v is from about 1 to about 6, in embodiments about 2 or 3, and w is from about 1 to about 12, in embodiments from about 1 to about 6.

Suitable B⁸ groups include alkylene, oxaalkylene and oligo-oxaalkylene groups of up to about 12 carbon atoms. In one embodiment B⁸ and K² contain together up to about 12 carbon atoms.

Particular examples of comonomers bearing a reactive group include chloromethylstyrene, methacrylic acid, 2-aminoethylmethacrylate, 2,3-epoxypropyl methacrylate, 3-chloro 2-hydroxypropylmethacrylate, 2-methacryloyloxy-ethyl dichlorotriazine, 3-chloro-2-hydroxy-propylmethacrylamide and glycidyl methacrylate and reactive methacrylate esters containing the group HetC(O)O— in which (Het) is a heterocycle ring, for example benzotriazole or imidazole and reactive methacrylate esters containing a group R¹⁶OC(O)— in which R¹⁶ is succinimide or pentafluorophenyl group.

Some suitable comonomers bearing reactive groups include 2-aminoethyl-methacrylate and 3-chloro-2-hydroxypropylmethacrylate.

Comonomers bearing a reactive group capable of binding covalently to a surface, such as those of formula (X) or (XI), are commercially available or may be prepared by conventional techniques using known reactions.

Comonomers of formula (X), which are dichlorotriazine monomers may be prepared in known manner for example by reacting a substituted hydroxy-alkyl(alk)acrylate or aminoalkyl(alk)acrylate with trichlorotriazine in a suitable solvent and in the presence of a base.

Comonomers of formula (XI) which are reactive methacrylate esters in which the ester groups contains an imidazole group may be prepared in known manner by reacting a substituted hydroxyalkyl(alk)acrylate (e.g. 2-hydroxyethyl-(meth)acrylate), polyethylene-oxide(meth)acrylate or polypropylene oxide (meth)acrylate with 1,1-carbonyl-diimidazole in a dry solvent. Analogous known methods may be used to prepare succinimide and pentafluorophenyl methacrylate esters of formula (X), by reaction with a reactive ester, acid halide or acid anhydride.

Where comonomers containing a reactive group are used to bind a copolymer to a surface by covalent bonding, it will be appreciated that not all of the reactive groups need necessarily bind to surface reactive groups and that groups not so bound may participate in other chemistry. Such groups may in particular provide points for the attachment of moieties such as ligands to the phosphorylcholine, which may be especially useful when the phosphorylcholine is used to form a composition of the present disclosure suitable for use in coating a substrate.

Comonomers containing a reactive group, such as compounds of formula (X) and (XI) may be used as comonomers containing crosslinkable groups, which react with other crosslinkable groups, rather than a monomer which bind covalently to a surface.

Where comonomers containing a reactive group are used to provide such crosslinkable groups then the crosslinkable groups and/or the copolymerization conditions will be chosen so that they will not crosslink when the comonomers are copolymerized; thus the polymerization product will be an uncrosslinked linear copolymer which may be subsequently crosslinked after coating the copolymer onto a surface so as to improve the stability of the coating. When such crosslinking between linear polymer chains is employed the crosslinkage may be formed either between two such crosslinkable groups or between a crosslinkable group and a non-inert group in a diluent comonomer residue. Such a crosslinkage may be formed either by direct reaction of the groups forming the crosslinkage or by reaction of these groups with a reactive bridging molecule for example a reactive gas, such as ammonia.

Residues of such comonomers may therefore be present in phosphorylcholines utilized in forming compositions of the present disclosure which are used to coat hydrophobic surfaces and containing residues of a monomer containing a group bearing a center of permanent positive charge which is of formula (VA), (VB) or (VC) or a comonomer containing an alkyl, fluoroalkyl or siloxane group, which is of formula (VII) or (VIII). Similarly residues of such comonomers may also be present in phosphorylcholines designed to bind to a surface by ionic interaction and which contains residues of a compound of formula (XIII) or (XIV) as defined below.

Suitable reactive comonomers which may be used to crosslink the comonomer, rather than provide covalent binding to the surface, are those of formula (X) or (XI) in which Q², or Q⁴ contains a crosslinkable cinnamyl, epoxy, —CHOHCH₂Hal (in which Hal is a halogen atom), methylol, silyl, an ethylenically unsaturated crosslinkable group, such as an acetylenic, diacetylenic, vinylic or divinylic group, or an acetoacetoxy or chloroalkyl sulfone, in embodiments chloroethyl sulfone, group.

Particular examples of comonomers bearing a group capable of crosslinking include methacrolein, cinnamyl methacrylate, 2,3-epoxypropyl methacrylate, 3-chloro-2-hydroxypropyl methacrylate, hydroxymethyl methacrylamide, 3-(trimethoxysilyl)propyl methacrylate, 2-acetoacetoxyethyl methacrylate, 3-(vinylbenzyl)-2-chloroethyl sulfone.

When a phosphorylcholine of the present disclosure, contains crosslinkable groups, it may be added to a composition of the present disclosure and coated on a substrate in substantially uncrosslinked form. After coating, crosslinking of crosslinkable groups may be performed to increase the strength and stability of the coating.

In other embodiments, comonomers which may be utilized in forming phosphorylcholines may possess an ionic group. Such comonomers bearing an ionic group may be of general formula (XII)

Y²—B⁹-Q⁵ (XII)

where Y² is an ethylenically unsaturated polymerizable group selected from

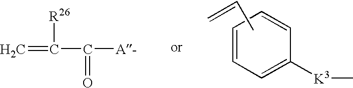

where R²⁶ is hydrogen or C₁-C₄ alkyl; A″ is —O— or —NR²⁷—, wherein R²⁷ is hydrogen or a C₁-C₄ alkyl group or R²⁷ is a group —B⁹-Q⁵;

B⁹ is a valence bond, a straight or branched alkylene, oxaalkylene or oligo-oxaalkylene group;

K³ is a group —(CH₂)ₓOC(O)—, —(CH)ₓC(O)O—, —(CH₂)ₓOC(O)O—, —(CH₂)ₓNR²⁸—, —(CH₂)ₓNR²⁸C(O)—, —(CH₂)ₓC(O)NR²⁸—, —(CH₂)ₓNR²⁸C(O)O—, —(CH₂)ₓOC(O)NR²⁸—, —(CH₂)ₓNR²⁸C(O)NR²⁸— (in which, the groups R²⁸ are the same or different), —(CH₂)ₓO—, —(CH₂)ₓSO₃—, a valence bond (optionally in combination with B⁹) and x is from 1 about to about 12 and R²⁸ is hydrogen or a C₁-C₄ alkyl group;

Q⁵ is an ionic group capable of binding to a surface by ionic interaction.

Suitable comonomers of formula (XII) are therefore those of formula (XIII) and (XIV):

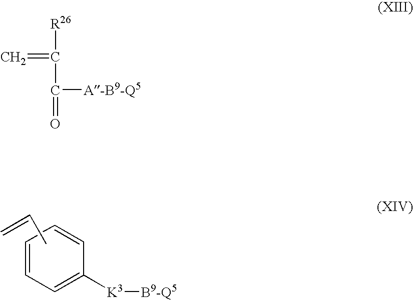

wherein: R²⁶, A″, B⁹, K³ and Q⁵ are as defined in relation to formula (XII) above.

In embodiments, in the compounds of formula (XIII), R²⁶ may be hydrogen, methyl or ethyl, in embodiments methyl, so that the compound of formula (XIII) may be an acrylic acid, methacrylic acid or ethacrylic acid derivative.

In the compounds of formula (XIV), K³ may, for instance, be a valence bond. Where K³ is a group, then x may be from about 1 to about 6, in embodiments about 1, 2 or 3, in some cases x may be about 1. When K³ is a group —(CH₂)ₓNR²⁶—, —(CH₂)ₓOC(O)NR²⁶— (CH₂)ₓNR²⁶C(O)O—, —(CH₂)ₓNR²⁶C(O)—, —(CH₂)ₓC(O)NR²⁶— or —(CH₂)ₓNR²⁶C(O)NR²⁶— then R²⁶ may be hydrogen, methyl or ethyl, in some embodiments hydrogen.

In the compounds of formula (XIV), the vinyl group may be para to the group —K³—B⁹-Q⁵.

In embodiments, B⁹ may be:

an alkylene group of formula —(CR²⁹₂)_{y}—, wherein the groups —(CR²⁹₂)— are the same or different, and in each group —(CR²⁹₂)— the groups R²⁹ are the same or different and each group R²⁹ is hydrogen or C₁₋₄ alkyl, in embodiments hydrogen, and y is from about 1 to about 12, in embodiments from about 1 to about 6;

an oxaalkylene group such as alkoxyalkyl having from about 1 to about 6 carbon atoms in each alkyl moiety; or

an oligo-oxaalkylene group of formula —[(CR³⁰₂)_{yy}O]ₓₓ(CR³⁰₂)_{yy}— where the groups —(CR³⁰₂)— are the same or different and in each group —(CR³⁰₂)— the groups R³⁰ are the same or different and each group R³⁰ is hydrogen or C₁₋₄ alkyl, in embodiments hydrogen, and yy is from about 1 to about 6, in embodiments about 2 or 3, and xx is from about 1 to about 12, in embodiments from about 1 to about 6.

Suitable B⁹ groups include alkylene, oxaalkylene and oligo-oxaalkylene groups of up to about 12 carbon atoms.

Particular examples of the B⁹ groups include —CH₂—, —CH₂CH₂— and —(CH₂)₆—.

The Q⁵ group may be either anionic or cationic depending upon the surface to be coated. Where the surface has a cationic surface charge, the Q⁵ group may be anionic and may, for example, be a carboxylate, sulfonate, hydrogen phosphate or phosphate group. Where the surface has an anionic surface charge, the Q⁵ group may be cationic and be, for example, —NR³¹₃⊕ in which each group R³¹ is the same or different, and may be hydrogen or alkyl of from about 1 to about 6 carbon atoms, two of which groups R³¹ may together from a heterocyclic ring containing from about 5 to about 7 atoms, in embodiments hydrogen or methyl, a group N ⊕ Het, where Het is an unsaturated heterocyclic group such as pyridyl, substituted or unsubstituted by one or more alkyl groups of from about 1 to about 4 carbon atoms, or a group —PR³²₃⊕ in which each group R³² is the same or different and is hydrogen or alkyl of from about 1 to about 6 carbons atoms, two of which groups R³² may together form a heterocyclic ring containing from about 5 to about 7 atoms, in embodiments methyl.

Particular examples of comonomers bearing an ionic group include acrylic acid, methacrylic acid, 2-sulfoethyl methacrylate, 2-methacryloyloxyethyl phosphate, p-styrene sulfonic acid, 2-(methacryloyloxyethyl)trimethylammonium chloride, 3-aminopropyl methacrylamide, vinylbenzyl trimethylammonium chloride.

Comonomers bearing a group capable of binding a polymer to a surface by ionic interaction, such as those of formula (XIII) and (XIV) are commercially available or may be prepared by conventional techniques using known reactions.

In addition to the residues of monomers containing a group bearing a center of permanent positive charge and/or the residues of comonomers containing a group bearing a center of permanent positive charge and comonomers which are capable of binding to a surface, the phosphorylcholines utilized in the compositions of the present disclosure may also possess residues of a diluent comonomer.

Such diluent comonomers may be used to give the phosphorylcholine utilized in the compositions of the present disclosure the desired physical and mechanical properties. They may be of any known conventional radical polymerizable, in embodiments ethylenically unsaturated, type compatible with other comonomer(s).

Particular examples of diluent comonomers include alkyl(alk)acrylates possessing from about 1 to about 4 carbon atoms in the alkyl group of the ester moiety, such as methyl (alk)acrylate; a dialkylamino alkyl(alk)acrylate, in embodiments containing from about 1 to about 4 carbon atoms in each alkyl moiety of the amine and from about 1 to about 4 carbon atoms in the alkylene chain, e.g. 2-(dimethylamino)ethyl (alk)acrylate; an alkyl (alk)acrylamide containing from about 1 to about 4 carbon atoms in the alkyl group of the amide moiety; a hydroxyalkyl (alk)acrylate, in embodiments containing from about 1 to about 4 carbon atoms in the hydroxyalkyl moiety, e.g. a 2-hydroxyethyl (alk)acrylate; or a vinyl monomer such as an N-vinyl lactam, in embodiments containing from about 5 to about 7 atoms in the lactam ring, for instance vinyl pyrrolidone; styrene or a styrene derivative which for example is substituted on the phenyl ring by one or more alkyl groups containing from about 1 to about 6, in embodiments from about 1 to about 4, carbon atoms, and/or by one or more halogen, such as fluorine atoms, e.g. (pentafluorophenyl)styrene.

Other suitable diluent comonomers include polyhydroxyl, for example sugar, (alk)acrylates and (alk)acrylamides in which the alkyl group contains from about 1 to about 4 carbon atoms, such as sugar acrylates, methacrylates, ethacrylates, acrylamides, methacrylamides and ethacrylamides. Suitable sugars include glucose and sorbitol. Particularly suitable diluent comonomers include methacryloyl glucose or sorbitol methacrylate.

Further diluents which may be mentioned include polymerizable alkylenes, in embodiments of from about 2 to about 4 carbon atoms, such as ethylene, dienes such as butadiene, alkylene anhydrides such as maleic anhydride, and cyano-substituted alkylenes, such as acrylonitrile.

Diluent comonomers may be obtained by conventional known methods.

Of the above diluent comonomers some are inert and act simply to modify the physical and mechanical properties of copolymers, such as phosphorylcholines, containing them. Others, and in particular the hydroxyalkyl(alk)acrylates and polyhydroxyl (alk)acrylates, have a reactive role in addition to simply modifying physical and mechanical properties. Such comonomers contain functional groups, such as hydroxyl groups, which may react with a crosslinking group or may react with reactive groups in other molecules to attach them to the copolymer.

It will also be appreciated that alkyl(alk)acrylates containing about 6 or more carbon atoms in the alkyl group may be regarded as either diluent comonomers or comonomers capable of binding a phosphorylcholine-containing polymer to a surface by physisorption. It should be noted that a copolymer which contains such a diluent comonomer and a reactive comonomer capable of reacting at a surface to provide covalent binding to a surface may be used to coat a hydrophilic surface, the reactive comonomer providing binding to the surface and the diluent modifying physical and mechanical properties. However, such a copolymer may also be to coat hydrophobic surfaces, in which the “diluent” monomer will act as a comonomer capable of binding to the surface by physisorption and the comonomer capable of covalent binding will act as a crosslinkable comonomer.

Phosphorylcholines useful in the compositions of the present disclosure may be prepared by methods within the purview of those skilled in the art. In embodiments, these phosphorylcholines, sometimes referred to herein as phosphorylcholine-containing polymers or copolymers, may be prepared by:

a) copolymerizing a comonomer containing a group bearing a center of permanent positive charge, in embodiments a zwitterionic group, a comonomer containing a group capable of stably binding the polymer to a surface and optionally a diluent and/or crosslinkable comonomer; or

b) polymerizing a monomer containing a group containing a group bearing a center of permanent positive charge, in embodiments a zwitterionic group, and a group capable of stably binding the polymer to a surface and optionally further comonomer containing a group capable of stably binding the polymer to the surface and a diluent and/or a crosslinkable comonomer.

Any conventional technique may be used for polymerization, including thermal or photochemical polymerization. Where comonomers capable of producing crosslinking in the coated polymer film are present, the polymerization conditions may be set such that crosslinking does not occur during polymerization. Thus, for example, actinic radiation may not be used to prepare a polymer containing a comonomer which can form crosslinks by exposure to actinic radiation.

For thermal polymerization, a temperature from about 40 to about 100° C., in embodiments from about 50 to about 80° C., may be used. For photochemical polymerization actinic radiation such as gamma, ultraviolet (U.V.), visible, or microwave radiation may be used. In some embodiments, U.V. radiation of a wavelength from about 200 to about 400 nm is used.

The polymerization is generally performed in a reaction medium, which is for instance a solution or dispersion using as a solvent for example acetonitrile, dimethyl formamide, chloroform, dichloromethane, ethyl acetate, dimethyl sulfoxide, dioxane, benzene, toluene, tetrahydrofuran, or where the polymer does not contain groups which react with protic solvents, water or an alkanol containing from about 1 to about 4 carbon atoms, e.g. methanol, ethanol or propan-2-ol. Alternatively, a mixture of any of the above solvents may be used.

The polymerization may be carried out in the presence of one or more polymerization initiators, such as benzoyl peroxide, 2,2′-azo-bis(2-methylpropionitrile) or benzoin methyl ether. Other polymerization initiators which may be used are within the purview of those skilled in the art and include, for example, those disclosed in “Polymer Handbook”, 3rd edition, Ed. J. Brandrup and E. H. Immergut, Pub. Wiley-Interscience, New York, 1989.

Generally the copolymerization is performed for a period from about 1 to about 72 hours, in embodiments from about 8 to about 48 hours, in some cases from about 16 to about 24 hours, and under an inert atmosphere of for example nitrogen or argon. The polymer may be purified by dialysis, precipitation in a non-solvent (e.g. diethyl ether or acetone) or ultrafiltration. The resulting polymer is generally dried under vacuum, e.g. for a period of time from about 5 to about 72 hours and has a molecular weight from about 10,000 to about 10 million, in embodiments from about 20,000 to about 1 million.

The precise proportion and nature of the various comonomers used to prepare a copolymer according to the present disclosure including residues of a comonomer containing a group bearing a center of permanent positive charge and a comonomer containing a group capable of stably binding the polymer to a surface may be adjusted to provide a copolymer which is particularly suitable for coating a particular surface. Thus the proportion of comonomer containing a group capable of stably binding the polymer to a surface may be adapted to provide efficient physisorption at a particular hydrophobic surface, to correspond to the number of functional groups at a particular surface or to provide efficient binding by ionic interaction with a particular surface. Similarly the proportion of the comonomer containing a group bearing a center of permanent positive charge and of diluent and/or crosslinkable comonomer may be adapted to provide the desired biocompatibility and physical and mechanical properties. It will be appreciated that to obtain the desired combination of properties more than one type of comonomer containing a group bearing a center of permanent positive charge, comonomer containing a group capable of stably binding the polymer to a surface or crosslinkable and/or diluent comonomer may be used.

Similarly, in polymers possessing residues of a monomer containing a group bearing a center of permanent positive charge and a group capable of stably binding the polymer to a surface, the nature of these groups may be adjusted to provide the desired biocompatibility and efficient binding at a particular surface, as well as desired physical and mechanical properties. Where, in addition, a diluent and/or crosslinkable comonomer is used the nature of the diluent and/or crosslinkable comonomer and the proportions of the comonomers may be likewise adjusted. It will again be appreciated that to obtain the desired combination of properties more than one type of monomer containing a group bearing a center of permanent positive charge and a group capable of stably binding the polymer to a surface and/or more than one type of crosslinkable and/or diluent comonomer may be used.

The monomer composition which is subjected to polymerization to provide a polymer according to the present disclosure includes a minimum of about 0.01%, in embodiments about 1%, in yet other embodiments about 5% by weight of monomer or monomers containing a group bearing a center of permanent positive charge and a maximum of about 99.9%, in embodiments about 99%, in yet other embodiments about 95% by weight of other monomer or monomers. Such other monomer or monomers may be a monomer or monomers containing a group capable of stably binding the polymer to a surface, a diluent monomer or monomers and/or a crosslinkable monomer or monomers.

The monomer composition further includes a minimum of about 0.01%, in embodiments about 1%, in yet other embodiments about 5% by weight of monomer or monomers containing a group capable of stably binding the polymer to a surface and a maximum of about 99.9%, in embodiments about 99%, in yet other embodiments about 95% by weight of other monomer or monomers. Such other monomer or monomers may be a monomer or monomers containing a group bearing a center of permanent positive charge, a diluent monomer or monomers and/or a crosslinkable monomer or monomers.

It will be appreciated that where at least some of the monomer or monomers containing a group bearing a center of permanent positive charge also contains a group capable of stably binding the polymer to a surface, at least a proportion of the content of both these groups is provided by the same monomer. In such a case the polymer may be a homopolymer of a monomer containing both these groups.

Where the polymer is to bind to a surface by physisorption then the monomer composition may include no more than about 95%, in embodiments no more than about 90% and in some cases no more than about 80% by weight of monomer or monomers containing an alkyl, fluoroalkyl or siloxane group which is capable of binding the polymer to a surface by physisorption and which does not also contain a group bearing a center of permanent positive charge, the balance of the composition being monomer or monomers containing a group bearing a center of permanent positive charge, diluent monomer or monomers and/or crosslinkable monomer or monomers. Such a composition typically includes up to about 50% by weight of diluent comonomer or comonomers. Where diluent comonomer is present, it may include at least about 1%, in embodiments about 5%, by weight of the total comonomer composition. Where present, crosslinkable comonomer or comonomers generally are present in an amount from about 0.1% to about 20% by weight of the total comonomer composition.

Where different comonomers are used to provide the center of permanent positive charge and the physisorption, then the molar ratio in the copolymer of comonomer residues bearing a center of permanent positive charge to comonomer residues containing an alkyl, fluoroalkyl or siloxane group capable of binding the polymer to a surface by physisorption may be from about 5:95 to about 80:20, in embodiments from about 10:90 to about 50:50. In addition the copolymer may include from about 5% to about 50%, in embodiments from about 10% to about 25%, by mole residues of diluent monomer and/or from about 0.1 to about 20%, in embodiments from about 1% to about 10%, by mole residues of crosslinkable comonomer, provided that where residues of both diluent and crosslinkable comonomer are present, they do not exceed in combination about 50%, in embodiments about 35% by mole.

Where the polymer is to bind covalently to a surface then the monomer composition generally includes no more than about 25%, in embodiments up to about 20% and in some cases up to about 15% by weight of monomer or monomers containing a group capable of binding the polymer to a surface covalently and which does not also contain a group bearing a center of permanent positive charge; the balance of the composition being monomer or monomers containing a group bearing a center of permanent positive charge, and optionally diluent monomer or monomers. Such a composition typically includes up to about 95%, in embodiments up to about to 90%, by weight of diluent comonomer or comonomers. Where diluent comonomer is present, it may include at least about 5%, in embodiments about 10%, by weight of the total comonomer composition.

The molar ratio in the copolymer of comonomer residues bearing a center of permanent positive charge to comonomer residues containing a reactive group capable of binding the polymer to a surface by covalent bonding may be from about 5:95 to about 95:5, in embodiments from about 50:50 to about 90:10. In addition, the copolymer may include from about 5% to about 50%, in embodiments from about 10% to about 25%, by mole residues of diluent monomer and/or from about 0.1% to about 20%, in embodiments from about 1% to about 10%, by mole residues of crosslinkable comonomer, provided that where residues of both diluent and crosslinkable comonomer are present, they do not exceed in combination about 50%, in embodiments about 35% by mole.

Where the polymer is to bind to a surface by ionic interaction, then the molar ratio in the copolymer of comonomer residues bearing a center of permanent positive charge to comonomer residues containing an ionic group capable of binding the polymer to a surface by ionic interactions may be from about 5:95 to about 95:5, in embodiments from about 50:50 to about 90:10. In addition, the copolymer may include from about 5% to about 50%, in embodiments from about 10% to about 25%, by mole residues of diluent monomer and/or from about 0.1% to about 20%, in embodiments from about 1% to about 10%, by mole residues of crosslinkable comonomer, provided that where residues of both diluent and crosslinkable comonomer are present, they do not exceed in combination about 50%, in embodiments about 35% by mole.

In addition the monomer or comonomer composition may include further components such as a polymerization initiator, chain transfer agent, acid, base, surfactant, emulsifier or catalyst of conventional type each in an amount from about 0.1% to about 5%, in embodiments from about 0.2% to about 3% and in some cases about 0.5%, by weight each relative to the total weight of the monomers.

In embodiments, suitable phosphorylcholines include those commercially available as PC 1059, PC 1036, PC 1062, PC 2028, PC 1071, PC 1015 and/or PC 2083 from Biocompatibles Limited (Middlesex, UK).

In addition to the copolymer(s) and phosphorylcholine(s) described above, compositions in accordance with the present disclosure may also include a fatty acid component, such as a fatty acid or a fatty acid salt or a salt of a fatty acid ester. Suitable fatty acids may be saturated or unsaturated, and may include higher fatty acids having more than about 12 carbon atoms. Suitable saturated fatty acids include, for example, stearic acid, palmitic acid, myristic acid and lauric acid. Suitable unsaturated fatty acids include oleic acid, linoleic acid, and linolenic acid. In addition, an ester of fatty acids, such as sorbitan tristearate or hydrogenated castor oil, may be used.

Suitable fatty acid salts include the polyvalent metal ion salts of C₆ and higher fatty acids, particularly those having from about 12 to about 22 carbon atoms, and mixtures thereof. Fatty acid salts including the calcium, magnesium, barium, aluminum, and zinc salts of stearic, palmitic and oleic acids may be useful in some embodiments of the present disclosure, that is, calcium stearate, magnesium stearate, barium stearate, aluminum stearate, zinc stearate, calcium palmitate, magnesium palmitate, barium palmitate, aluminum palmitate, zinc palmitate, calcium oleate, magnesium oleate, barium oleate, aluminum oleate, and zinc oleate. Some useful salts include commercial “food grade” calcium stearate which contains a mixture of about one-third C₁₆ and two-thirds C₁₈ fatty acids, with small amounts of the C₁₄ and C₂₂ fatty acids.

Suitable salts of fatty acid esters which may be included in the compositions of the present disclosure include calcium, magnesium, aluminum, barium, or zinc stearoyl lactylate; calcium, magnesium, aluminum, barium, or zinc palmityl lactylate; and/or calcium, magnesium, aluminum, barium, or zinc oleyl lactylate. In embodiments; calcium stearoyl-2-lactylate (such as the calcium stearoyl-2-lactylate commercially available under the tradename VERV from American Ingredients Co., Kansas City, Mo.) may be utilized. Other fatty acid ester salts which may be utilized include those selected from the group consisting of lithium stearoyl lactylate, potassium stearoyl lactylate, rubidium stearoyl lactylate, cesium stearoyl lactylate, francium stearoyl lactylate, sodium palmityl lactylate, lithium palmityl lactylate, potassium palmityl lactylate, rubidium palmityl lactylate, cesium palmityl lactylate, francium palmityl lactylate, sodium oleyl lactylate, lithium oleyl lactylate, potassium oleyl lactylate, rubidium oleyl lactylate, cesium oleyl lactylate, and francium oleyl lactylate.

As noted above, the phosphorylcholines may be combined with the copolymer(s) and, together, they may form the minor component of the composition of the present disclosure, while the fatty acid component may form the predominant component of the composition of the present disclosure. Thus, in embodiments, the copolymers may be present in the compositions of the present disclosure in amounts from about 1 to about 9 percent by weight of the composition, in embodiments from about 2 to about 7 percent by weight of the composition. The phosphorylcholine-containing polymers may be present in amounts from about 0.5 to about 9 percent by weight of the composition, in embodiments from about 1 to about 5 percent by weight of the composition. The fatty acid component may thus be present in amounts from about 0.5 to about 5 percent by weight of the composition, in other embodiments from about 1 to about 3 percent by weight of the composition.

The copolymer, the fatty acid component, and the phosphorylcholine-containing polymer are non-toxic; a combination of the three is non-toxic as well.

Methods for combining the copolymers, phosphorylcholine-containing polymers, and fatty acid components to form compositions of the present disclosure are within the purview of those skilled in the art and include, for example, mixing, blending, and the like.

In embodiments, a fatty acid ester such as calcium stearoyl lactylate may be utilized in forming a composition of the present disclosure. Since such fatty acid esters are soluble, coatings fabricated from compositions of the present disclosure including such fatty acid esters, phosphorylcholines, and copolymers, including the caprolactone containing copolymers described above, may be applied as solutions to articles including surgical articles by processes including spraying, dipping, and the like.

In embodiments, the compositions of the present disclosure may be applied in a solution utilizing any suitable solvent within the purview of those skilled in the art. Such solvents include, for example, methylene chloride, chloroform, N,N-dimethylformamide, N-methylpyrrolidone, methanol, ethanol, n-propanol, isopropanol, hexane, heptane, cyclohexane, tetrahydrofuran (THF), dipropyl ether, dioxolane, methyl ethyl ketone, methyl isobutyl ketone, toluene, xylene, acetonitrile, ethyl acetate, butyl acetate, combinations thereof, and the like.

In embodiments, coating suspensions may be utilized wherein the composition of the present disclosure is combined with multiple solvents, including any solvent described above. In embodiments, suitable solvents for forming coating suspensions include, but are not limited to, lower alcohols including those having from about 1 to about 6 carbon atoms such as methanol, ethanol, n-propanol, isopropanol (IPA), n-butanol, combinations thereof, and the like; hexanes including hexane, cyclohexane, combinations thereof, and the like; chlorinated solvents such as methylene chloride, chloroform, chlorobenzene, 1,2-dichloroethane (also known as ethylene dichloride), dichlorobenzene, combinations thereof, and the like. As noted above, in some embodiments multiple solvents may be utilized, including any combination of the foregoing solvents.

For example, the composition of the present disclosure can be applied as a coating by any suitable process, for example, by passing a medical device through a solution of the composition, past a brush or other coating solution applicator, or past one or more spray nozzles dispensing a solution possessing a composition of the present disclosure for use as a coating. The article wetted with the coating solution may then be subsequently passed through, or held in, a drying oven for a time and at a temperature sufficient to vaporize and drive off the solvent. If desired, the coating composition can optionally contain additional components, e.g., dyes, antibiotics, antiseptics, growth factors, anti-inflammatory agents, and the like.

Where applied as a coating, the composition of the present disclosure may be applied in a continuous, that is, a single step. In other embodiments, the composition of the present disclosure may be applied in discontinuous or semi-continuous steps, that is, a multi-step process.

The composition of the present disclosure may be applied as a coating in a single layer or, in embodiments, the composition of the present disclosure may be applied as a coating in multiple layers and/or as overcoats to other preexisting coatings on an article. In some embodiments, a coating of the present disclosure may include the three components described herein applied sequentially. For example, a copolymer, such as a caprolactone containing copolymer, may be combined with a fatty acid component, and the combination of the two may then be utilized to form a medical device or a coating thereon. The phosphorylcholines described above may then be applied as a separate coating to the device made from, or already possessing a coating including, the caprolactone containing copolymer combined with the fatty acid component.

The compositions of the present disclosure may find many uses in the formation of medical devices and coatings thereon. In embodiments, surgical articles can be manufactured from, or coated with, the compositions described herein. Suitable medical devices include, but are not limited to, clips and other fasteners, staples, sutures, pins, screws, prosthetic devices, wound dressings, drug delivery devices, anastomosis rings, and other implantable devices. Fibers can be made from, or coated with, the compositions of the present disclosure. In embodiments, fibers made or coated with the compositions of the present disclosure may be knitted or woven with other fibers, either absorbable or non-absorbable fibers, to form textiles. The fibers also can be made into non-woven materials to form fabrics, such as meshes and felts.

In embodiments, the fatty acid component combined with phosphorylcholines and copolymers can advantageously be mixed to form a composition (with the fatty acid component as the predominant component thereof) useful in coating surgical sutures.

While the coating composition herein can be applied to any type of suture, it may, in some embodiments, be useful for coating a braided suture, including those disclosed in U.S. Pat. No. 5,019,093, the entire disclosure of which is incorporated by reference herein. In embodiments, useful compositions for coating braided multifilament sutures may include at least about 52 percent fatty acid component, the remainder being the phosphorylcholine in combination with the copolymer. Applied to a suture, the coating composition results in advantageous improvement in one or more properties of the suture, e.g., knot security, surgeon's throw, lubricity, knot run down, and/or knot repositioning.

The amount of coating composition applied to a braided suture will vary depending upon the structure of the suture, e.g., the number of filaments, tightness of braid or twist, the size of the suture, and its composition. Suitable coating levels range from about 0.3% to about 10% by weight of the suture, with about 0.5% to about 5% by weight of the suture being useful in some embodiments.

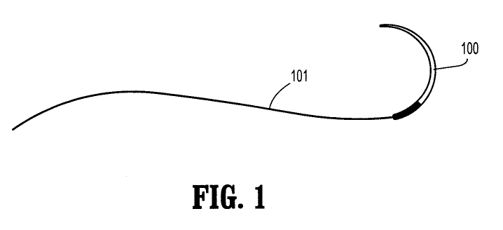

The coated suture, suture 101, may be attached to a surgical needle 100 as shown in FIG. 1 by methods within the purview of those skilled in the art. Wounds may be sutured by passing the needled suture through tissue to create wound closure. The needle may then be removed from the suture and the suture tied. The coating advantageously enhances the surgeon's ability to pass the suture through tissue as well as to increase the ease and security with which he/she can tie the suture.

Sutures possessing coatings of the present disclosure possess excellent handling properties and knot security, which may, in embodiments, be determined using tie board testing and similar methods within the purview of those skilled in the art. Sutures possessing coatings of the present disclosure also possess excellent knot rundown and reposition, and both wet and dry handling characteristics. Methods for determining these properties are within the purview of those skilled in the art, including the use of apparatus and methods from Instron Corporation (Norwood, Mass.).

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims. Unless specifically recited in a claim, steps or components of claims should not be implied or imported from the specification or any other claims as to any particular order, number, position, size, shape, angle, color, or material.

What is claimed is:

1. A coating for a medical device comprising:
a copolymer comprising a predominant amount of epsilon-caprolactone and a minor amount of at least one other copolymerizable monomer;
at least one phosphorylcholine; and
a fatty acid component selected from the group consisting of salts of a fatty acid and salts of a fatty acid ester.

2. The coating of claim 1, wherein the copolymer comprises from about 70 to about 98 weight percent epsilon-caprolactone derived units.

3. The coating of claim 1, wherein the copolymer comprises from about 80 to about 95 weight percent epsilon-caprolactone derived units.

4. The coating of claim 1, wherein the at least one other copolymerizable monomer comprises glycolide.

5. The coating of claim 1, wherein the at least one phosphorylcholine comprises a phosphorylcholine selected from the group consisting of 2-methacryloyloxyethyl phosphorylcholine (MPC), 2-acryloyloxyethyl phosphorylcholine, and combinations thereof.

6. The coating of claim 1, wherein the at least one phosphorylcholine comprises a phosphorylcholine comprising a monomer selected from the group consisting of 2-(meth)acryloyloxyethyl-2′-(trimethylammonio)ethyl phosphate, 3-(meth)acryloyloxypropyl-2′-(trimethylammonio)ethyl phosphate, 4-(meth)acryloyloxybutyl-2′-(trimethylammonio)ethyl phosphate, 5-(meth)acryloyloxypentyl-2′-(trimethylammonio)ethyl phosphate, 6-(meth)acryloyloxyhexyl-2′-(trimethylammonio)ethyl phosphate, 2-(meth)acryloyloxyethyl-2′-(triethylammonio)ethyl phosphate, 2-(meth)acryloyloxyethyl-2′-(tripropylammonio)ethyl phosphate, 2-(meth)acryloyloxyethyl-2′-(tributylammonio)ethyl phosphate, 2-(meth)acryloyloxypropyl-2′-(trimethylammonio)ethyl phosphate, 2-(meth)acryloyloxybutyl-2′-(trimethylammonio)ethyl phosphate, 2-(meth)acryloyloxypentyl-2′-(trimethylammonio)ethyl phosphate, 2-(meth)acryloyloxyhexyl-2′-(trimethylammonio)ethyl phosphate, 2-(meth)acryloyloxyethyl-3′-(trimethylammonio)propyl phosphate, 3-(meth)acryloyloxypropyl-3′-(trimethylammonio)propyl phosphate, 4-(meth)acryloyloxybutyl-3′-(trimethylammonio)propyl phosphate, 5-(meth)acryloyloxypentyl-3′-(trimethylammonio)propyl phosphate, 6-(meth)acryloyloxyhexyl-3′-(trimethylammonio)propyl phosphate, 2-(meth)acryloyloxyethyl-4′-(trimethylammonio)butyl phosphate, 3-(meth)acryloyloxypropyl-4′-(trimethylammonio)butyl phosphate, 4-(meth)acryloyloxybutyl-4′-(trimethylammonio)butyl phosphate, 5-(meth)acryloyloxypentyl-4′-(trimethylammonio)butyl phosphate, 6-(meth)acryloyloxyhexyl-4′-(trimethylammonio)butylphosphate, and combinations thereof.

7. The coating of claim 1, wherein the salt of a fatty acid comprises a polyvalent metal ion salt of C₆ and higher fatty acids.

8. The coating of claim 7, wherein the salt of a fatty acid is selected from the group consisting of calcium stearate, magnesium stearate, barium stearate, aluminum stearate, zinc stearate, calcium palmitate, magnesium palmitate, barium palmitate, aluminum palmitate, zinc palmitate, calcium oleate, magnesium oleate, barium oleate, aluminum oleate, and zinc oleate.

9. The coating of claim 1, wherein the salt of a fatty acid ester comprises a salt of lactylate esters of C₁₀ or greater fatty acids.

10. The coating of claim 9, wherein the salt of lactylate esters of C₁₀ or greater fatty acids is selected from the group consisting of magnesium stearoyl lactylate, aluminum stearoyl lactylate, barium stearoyl lactylate, zinc stearoyl lactylate, calcium palmityl lactylate, magnesium palmityl lactylate, aluminum palmityl lactylate, barium palmityl lactylate, zinc palmityl lactylate, calcium oleyl lactylate, magnesium oleyl lactylate, aluminum oleyl lactylate, barium oleyl lactylate, and zinc oleyl lactylate.

11. The coating of claim 1, further comprising a solvent selected from the group consisting of methylene chloride, chloroform, N,N-dimethylfoiinamide, N-methylpyrrolidone, methanol, ethanol, n-propanol, isopropanol, hexane, heptane, cyclohexane, tetrahydrofuran, dipropyl ether, dioxolane, methyl ethyl ketone, methyl isobutyl ketone, toluene, xylene, acetonitrile, ethyl acetate, butyl acetate, and combinations thereof.

12. The coating of claim 1, wherein the components of the coating are applied separately in multiple layers.

13. The coating of claim 1, wherein the components of the coating are applied as a single layer.

14. The coating of claim 1, wherein the medical device is selected from the group consisting of clips, fasteners, staples, sutures, pins, screws, prosthetic devices, wound dressings, drug delivery devices, anastomosis rings, and implants.

15. A suture possessing the coating of claim 1, wherein the coating is present in an amount from about 0.3% to about 10% by weight of the suture.

16. A surgical suture coating comprising:
a copolymer comprising from about 70 to about 98 weight percent epsilon-caprolactone derived units and at least one other copolymerizable monomer comprising glycolide;
at least one phosphorylcholine selected from the group consisting of 2-methacryloyloxyethyl phosphorylcholine (MPC), 2-acryloyloxyethyl phosphorylcholine, and combinations thereof; and
a fatty acid component selected from the group consisting of salts of a fatty acid and salts of a fatty acid ester.

17. The surgical suture coating of claim 16, wherein the salt of a fatty acid is selected from the group consisting of calcium stearate, magnesium stearate, barium stearate, aluminum stearate, zinc stearate, calcium palmitate, magnesium palmitate, barium palmitate, aluminum palmitate, zinc palmitate, calcium oleate, magnesium oleate, barium oleate, aluminum oleate, and zinc oleate, and the salt of a fatty acid ester is selected from the group consisting of magnesium stearoyl lactylate, aluminum stearoyl lactylate, barium stearoyl lactylate, zinc stearoyl lactylate, calcium palmityl lactylate, magnesium palmityl lactylate, aluminum palmityl lactylate, barium palmityl lactylate, zinc palmityl lactylate, calcium oleyl lactylate, magnesium oleyl lactylate, aluminum oleyl lactylate, barium oleyl lactylate, and zinc oleyl lactylate.

18. A suture comprising:
a fiber;
a coating on at least a portion of the fiber, the coating comprising:
a copolymer comprising a predominant amount of epsilon-caprolactone and a minor amount of at least one other copolymerizable monomer;
at least one phosphorylcholine; and
a fatty acid component selected from the group consisting of salts of a fatty acid and salts of a fatty acid ester.

19. The suture of claim 18, wherein the copolymer comprises from about 70 to about 98 weight percent epsilon-caprolactone derived units and at least one other copolymerizable monomer comprising glycolide, the at least one phosphorylcholine comprises a phosphorylcholine selected from the group consisting of 2-methacryloyloxyethyl phosphorylcholine (MPC), 2-acryloyloxyethyl phosphorylcholine, and combinations thereof, the salt of a fatty acid is selected from the group consisting of calcium stearate, magnesium stearate, barium stearate, aluminum stearate, zinc stearate, calcium palmitate, magnesium palmitate, barium palmitate, aluminum palmitate, zinc palmitate, calcium oleate, magnesium oleate, barium oleate, aluminum oleate, and zinc oleate, and the salt of a fatty acid ester is selected from the group consisting of magnesium stearoyl lactylate, aluminum stearoyl lactylate, barium stearoyl lactylate, zinc stearoyl lactylate, calcium palmityl lactylate, magnesium palmityl lactylate, aluminum palmityl lactylate, barium palmityl lactylate, zinc palmityl lactylate, calcium oleyl lactylate, magnesium oleyl lactylate, aluminum oleyl lactylate, barium oleyl lactylate, and zinc oleyl lactylate.

20. The suture of claim 18, wherein the suture comprises a multi-filament suture and the coating is present in an amount from about 0.3% to about 10% by weight of the suture.
